(19) United States
(12) Patent Application Publication
(10) Pub. No.: US 2021/0068692 A1
(43) Pub. Date: Mar. 11, 2021

(54) DATA PROCESSING APPARATUS FOR ASSESSING ARRHYTHMIA OF A HEART

(71) Applicant: Alekseev Alekseev Siegle Röder GbR, München (DE)

(72) Inventors: Michail Alekseev, St. Petersburg (RU); Aleksandr Alekseev, St. Petersburg (RU); Reinhard Siegle, Augsburg (DE); Gunther Röder, München (DE)

(73) Assignee: Alekseev Alekseev Siegle Röder GbR, München (DE)

(21) Appl. No.: 16/640,290

(22) PCT Filed: Aug. 20, 2018
(86) PCT No.: PCT/EP2018/000407

§ 371 Date: Feb. 19, 2020

(30) Foreign Application Priority Data
Aug. 19, 2017 (EP) EP17001410

(51) Int. Cl. A61B 5/04; A61B 5/0452; A61B 5/0464; A61B 5/046; A61B 5/0468

(52) U.S. Cl. CPC A61B 5/04012; A61B 5/0452; A61B 5/0468; A61B 5/046; A61B 5/0464

(57) ABSTRACT

This invention concerns a data processing apparatus (12) for processing ST-segment slope information (ST1, ST2, STX) from an ECG signal from a myocardium of a heart of a human (1) or animal to generate data for one of—an assessment of a risk, a probability or a presence and—a diagnosis of a risk, a probability or a presence of a type of arrhythmia of the heart or a part of it, wherein the data processing apparatus (12) is configured to calculate at least two ST-segment slope values (STS1, STS2, STSX) wherein each of the at least two ST-segment slope values (STS1, STS2, STSX) belongs to ST-segments (ST) from different heart beats (HB1, HB2, HBX) of the same heart, and to derive a ST-segment slope deviation value (1DV, N1DV, S1DV, SN1DV) from at least two ST-segment slope values (STS1, STS2, STSX). The invention further concerns an assessment apparatus (14), and a method for providing data for an assessment and/or a diagnosis of a condition or a disease of at least a part of the myocardium.

This invention concerns a data processing apparatus for processing ST-segment slope information from an electrical signal from a myocardium of a heart of a human or animal, for quantitative assessment of the risk or the presence or the extent of arrhythmias of the heart or a part of it. Further, an assessment apparatus for assessing risk and/or presence and/or extent of arrhythmias of at least a part of a myocardium of a heart of an individual human or animal is provided. Furthermore, the invention concerns a method is for providing data for an assessment of the risk or the presence or the extent of arrhythmias of at least a part of the myocardium of the heart of an individual human or animal.

“Human or animal” preferably means in this context one single human or animal. This invention enables an assessment and/or a diagnose of the condition of a heart of a single individual.

In the field of cardiac electrophysiology electrocardiogram has been developed. The recording of cellular membrane currents revealed that the body surface ECG is the timed sum of the cellular action potentials in atria and ventricles. The clinical problem of sudden death caused by ventricular arrhythmias, most commonly caused by coronary obstruction, was recognized as early as the late nineteenth century. The problem was vexing and led to the development of pharmacologic and non pharmacologic therapies, including transthoracic defibrillators, cardiac massage, and implantable defibrillators.

The normal cardiac impulse is generated by pacemaker cells in sinoatrial node located at the junction of the right atrium and superior cava vena. This impulse is slowly transmitted through nodal tissue to the anatomically complex atria, where it is more rapidly conducted to the atrioventricular node (AVN) is reflected with the P wave of the ECG. There is a perceptible delay in conduction through the anatomically and functionally heterogeneous AVNs. The time needed for activation of the atria and the AVN delay is represented as the PR interval of the ECG. The AVN is the only electrical connection between the atria and ventricles in the normal heart. The electrical impulse emerges from the AVN and is transmitted to the His-Purkinje system, specifically the common bundle of His, then the left and right bundle branches, and hence to the Purkinje network, facilitating activation of ventricle muscle. Under normal circumstances, the ventricles are rapidly activated in a well-defined fashion that is determined by the course of Purkinje network and inscribes QRS complex. Recovery of electrical excitability occurs more slowly and is governed by the time of activation and duration of regional action potentials. The relative brevity of epicardial action potentials in the ventricle results in repolarization occurring first on the epicardial surface then proceeding to the endocardium, which inscribes a T wave normally of the same polarity as the QRS complex. The duration of activation and recovery is determined by the action potential duration represented by the QT interval, in the body surface ECG.

The gross anatomic features of the heart—the extracellular matrix and intramural vasculature—create both macro- and micro-anatomic barriers that are central to both the normal electrophysiology of the heart and clinically important arrhythmias.

Physicians and emergency department clinicians have a difficult task in prevention of arrhythmias and in detecting episodes of arrhythmias, especially fatal ones. Nowadays there is no technology or algorithm for quantitative and authentic criterion for assessing the risk or presence or extent of different types of arrhythmias, to forecast them and sometimes to detect a few types of arrhythmias in time.

Premature atrial contractions (PACs), also known as atrial premature complexes (APC) or atrial premature beats (APB), are a common cardiac dysrhythmia characterized by premature heartbeats originating in the atria. While the sinoatrial node typically regulates the heartbeat during normal sinus rhythm, PACs occur when another region of the atria depolarizes before the sinoatrial node and thus triggers a premature heartbeat. The exact cause of PACs is unclear; while several predisposing conditions exist, PACs commonly occur in healthy young and elderly people. Elderly people that get PACs usually don't need any further attention besides follow ups due to unclear evidence. PACs are often completely asymptomatic and may be noted only with Holter monitoring, but occasionally they can be perceived as a skipped beat or a jolt in the chest.

On an electrocardiogram (ECG), PACs are characterized by an abnormally shaped P wave. Since the premature beat initiates outside the sinoatrial node, the associated P wave appears different from those seen in normal sinus rhythm. Typically, the atrial impulse propagates normally through the atrioventricular node and into the cardiac ventricles, resulting in a normal, narrow QRS complex. However, if the atrial beat is premature enough, it may reach the atrioventricular node during its refractory period, in which case it will not be conducted to the ventricle and there will be no QRS complex following the P wave.

Premature junctional contractions (PJCs), also called atrioventricular junctional premature complexes or junctional extrasystole, are premature cardiac electrical impulses originating from the atrioventricular node of the heart or “junction”. This area is not the normal but only a secondary source of cardiac electrical impulse formation. These premature beats can be found occasionally in healthy people and more commonly in some pathologic conditions, typically in the case of drug cardiotoxicity, electrolyte imbalance, mitral valve surgery, and cold water immersion. If more than two such beats are seen, then the condition is termed junctional rhythm. On the surface ECG, premature junctional contractions will appear as a normally shaped ventricular complex or QRS complex, not preceded by any atrial complex or P wave or preceded by an abnormal P wave with a shorter distance or interval between the P wave and the QRS. Rarely, the abnormal P wave can follow the QRS.

A premature ventricular contraction (PVC)—also known as a premature ventricular complex, ventricular premature contraction (or complex or complexes) (VPC), ventricular premature beat (VPB), or ventricular extrasystole (VES)—is a relatively common event where the heartbeat is initiated by Purkinje fibers in the ventricles rather than by the sinoatrial node, the normal heartbeat initiator. The electrical events of the heart detected by the electrocardiogram (ECG) allow a PVC to be easily distinguished from a normal heart beat. Although a PVC can be a sign of decreased oxygenation to the heart muscle, often PVCs are benign and may even be found in otherwise healthy hearts.

A PVC may be perceived as a “skipped beat” or felt as palpitations in the chest. In a normal heartbeat, the ventricles contract after the atria have helped to fill them by contracting; in this way the ventricles can pump a maximized amount of blood both to the lungs and to the rest of the body. In a PVC, the ventricles contract first and before the atria have optimally filled the ventricles with blood, which means that circulation is inefficient. However, single beat PVC abnormal heart rhythms do not usually pose a danger and can be asymptomatic in healthy individuals.

Premature ventricular contractions can occur in a healthy person of any age, but are more prevalent in the elderly and in men. They frequently occur spontaneously with no known cause. Heart rate turbulence (HRT) is a phenomenon representing the return to equilibrium of the heart rate after a PVC. HRT parameters correlate significantly with mortality after myocardial infarction (heart attack). The following stimulants, conditions and triggers may increase your risk of the more frequent occurrence of premature ventricular contractions: Caffeine, tobacco and alcohol, Exercise, High blood pressure, Anxiety, Underlying heart disease, including congenital heart disease, coronary artery disease, heart attack, heart failure and a weakened heart muscle (cardiomyopathy), Male sex, Faster sinus rates, A bundle-branch block on 12-lead ECG. Holter monitoring is a far better method for diagnosis as it is continuous recording of the heart's rhythm over a period of 24 hours, or event monitoring which records non-continuously for 30 days or indefinitely. This increases the likelihood of a premature ventricular contraction occurring during the recording period and is therefore more useful in diagnosis. Another method of detection of PVCs is a portable electrocardiogram device known as an event recorder that can be carried around for home monitoring of the heart's activity. Both the Holter monitor and the event recorder can help to identify the pattern of a PVC.

The inventors suppose that, in case of polymorphic premature ventricular contractions, the nature of PVC is a result of metabolic problem in a corresponding part the myocardium, and as a result, it decreases the resting membrane potential of cardiomyocytes in one or more corresponding parts of the heart.

Atrial flutter (AFL) is a common abnormal heart rhythm that starts in the atrial chambers of the heart. When it first occurs, it is usually associated with a fast heart rate (100 or more heartbeats per minute), and is classified as a type of supra-ventricular tachycardia. Although this abnormal heart rhythm typically occurs in individuals with cardiovascular disease (e.g. high blood pressure, coronary artery disease, and cardiomyopathy) and diabetes mellitus, it may occur spontaneously in people with otherwise normal hearts. It is typically not a stable rhythm, and often degenerates into atrial fibrillation (AF). However, it does rarely persist for months to years. Atrial flutter is caused by a re-entrant rhythm in either the right or left atrium. The impact and symptoms of atrial flutter depend on the heart rate of the patient. There are two types of atrial flutter, the common type I and rarer type II. Type I atrial flutter, also known as common atrial flutter or typical atrial flutter, has an atrial rate of 240 to 340 beats per minute. Type II flutter follows a significantly different re-entry pathway to type I flutter, and is typically faster, usually 340-400 beats per minute.

The inventors suppose that the nature of AFL and AF is a result of a metabolic problem in an atrial muscle, and as a result, it decreases the resting membrane potential of the atrial cardiomyocytes.

All people with AF are initially in the category called first detected AF. These patients may or may not have had previous undetected episodes. If a first detected episode stops on its own in less than 7 days and then another episode begins, later on, the category changes to paroxysmal AF. Although patients in this category have episodes lasting up to 7 days, in most cases of paroxysmal AF the episodes will stop in less than 24 hours. If the episode lasts for more than 7 days, it is unlikely to stop on its own, and is then known as persistent AF.

Ventricular tachycardia (V-tach or VT) is a type of regular and fast heart rate that arises from improper electrical activity in the ventricles of the heart. Although a few seconds may not result in problems, longer periods are dangerous. Short periods may occur without symptoms or present with lightheadedness, palpitations, or chest pain. Ventricular tachycardia may result in cardiac arrest and turn into ventricular fibrillation. Ventricular tachycardia is found initially in about 7% of people in cardiac arrest.

Ventricular fibrillation (V-fib or VF) is when the heart quivers instead of pumping due to disorganized electrical activity in the ventricles. It is a type of cardiac arrhythmia. Ventricular fibrillation results in cardiac arrest with loss of consciousness and no pulse. This is followed by death in the absence of treatment. Ventricular fibrillation is found initially in about 10% of people in cardiac arrest.

The inventors suppose that the nature of VT (V-tach) and VF (V-fib) is a result of of a metabolic problem in a ventricle muscle or one or more parts of it, and, as a result, it decreases the resting membrane potential of ventricular cardiomyocytes in the heart or one or more parts of it.

A bundle branch block is a defect of the bundle branches of fascicles in the electrical conduction system of the heart. When a bundle branch or fascicle becomes injured by underlying heart disease, myocardial infarction, or cardiac surgery), it may cause to conduct electrical impulses appropriately. Depending on the anatomical location of the defect which leads to a bundle branch block, the blocks are further classified into right bundle and left bundle branch block. The left bundle branch block can be further sub classified into left anterior fascicular block and left posterior fascicular block. A bundle branch block can be diagnosed when the duration of the QRS complex on the ECG exceeds 120 ms. A right bundle branch block typically causes prolongation of the last part of the QRS complex, and may shift the heart's electrical axis slightly to the right. Left bundle branch block widens the entire QRS complex, and the most cases shifts the heart's electrical axis to the left. Some people with bundle branch block are born with this condition. Many other acquire it as a consequence of heart disease. People with bundle branch block may still be quite active, and many have nothing more remarkable than an abnormal appearance to their ECG. However, when bundle branch block are complex and diffuse in the bundle system, or associated with additional and significant ventricular muscle damage, they may be sign of serious underlying heart disease. In more severe cases, a pacemaker may be required to restore an optimal electrical supply to the heart muscle.

The inventors suppose that the nature of BBB is a result of a metabolic problem in a corresponding part is a result of metabolic problems in a corresponding part of the heart muscle and, as a result, it increases or even extremely hyperpolarizes the resting potential of the cardiomyocytes in a corresponding part of the heart.

Objective of this invention is to provide an apparatus and a method for data processing with the goal of assessing a risk, presence or extent of arrhythmia of a heart or a part of it. Assessment data as a basis for assessment shall be provided by a data processing apparatus and/or a data processing method.

The subject matter of this invention is defined by the features of independent claims. Preferred embodiments are defined by the dependent subclaims.

A data processing apparatus for generating data for assessment or diagnosis of a risk, probability and/or presence of different types of arrhythmias of a myocardium or a part of a myocardium is proposed.

A data processing apparatus for generating data for assessment of a risk, presence and/or extent of at least one type of arrhythmia of a heart or a part of it is proposed. Some relevant types of arrhythmia are described above. The apparatus processes ST-segment slope information of at least two ST-segment slopes which are commonly known as a part of a PQRST-complex. ST-segments are commonly known as a part of a PQRST-complex of an electrocardiogram.

It has been discovered that there is a correlation between temporal deviations of ST-segment slope and a risk, presence and extent of different types of arrhythmias of the heart or a part of it.

A “temporal deviation” means that information from at least two different heart beats is processed. Preferably, the information is acquired from the same source which can be the same location at the heart and/or the same or a neighbour electrode or line.

“Arrhythmias” of a heart or a part of it can mean in this context a presence of heart rate disturbance. “Arrhythmias” also can mean certain pathological or non-pathological states of a heart or a part of it providing the ST-segment of the myocardium and its slope. The state of the myocardium and, in consequence, of the heart is considered a result of the myocardial metabolism. From a result of a clinical study it seems clear that the myocardial metabolism and individual anatomical features of cardiac vessels are in close correlation with different types of arrhythmias. Thus, different heart diseases can be detected by different types of ST-segment slope deviation values, preferably from their presence and/or their quantities.

The inventors have found out that arrhythmias of a heart or a part of it do not only rely on the re-entry mechanism of development, but relies on a state of metabolism of myocardium or a part of it during ST-segment and on the influence of the state of metabolism on a resting membrane potential in atrias and ventricles. The state of depolarized ventricles which is presented by the ST-segment is affected by ischemic heart disease or other diseases or conditions of a heart. The inventors have further found out that a bundle branch block is a result of a metabolic disturbance in a corresponding part of the heart muscle and its rest potential.

ST-segment slope information can comprise an angle between isoline and ST-segment or an angle offset between ST-segments and arbitrary independent line in the ECG or a direction of another part of the PQRST-complex and ST-segment or a part or an average direction of it or a part of it, that can be derived from the respective part of waveform.

ST-segment slope information includes information about the slope that can be derived from ST-segment in the raw waveform in different ways. Particularly, a ST-segment slope value in an ECG signal can be calculated as an angle between an ST-segment or a characteristic part of it and zeroline or isoline and/or an angle between the ST-segment or a characteristic part of it and line fixed in amplitude-time scale and/or an angle between the ST-segment or a characteristic part of it and other parts of PQRST-complex and/or a ratio of the amplitude change to the time change between any two distinct points on a ST-segment and/or a difference between the initial amplitude of ST-segment or a part of it and the finite amplitude of ST-segment or a part of it and/or a function parameter of a function approximating or matching the ST-segment slope or a characteristic part of it, wherein the data processing apparatus is configured to approximate or describe ST-segment slope information with a mathematical function are relevant. ST-segment slope can have positive sign and/or negative sign.

Alternatively or additionally, a ST-segment slope value in an ECG signal can be defined as a difference between two values of ST-segment, preferably in a known time distance to each other or at characteristic points in the ST segment waveform, or a difference between or a ratio of two function values of an injective or bijective function of which the the argument is a value of ST-segment, respectively, preferably in a known time distance to each other or at characteristic points in the ST segment waveform, wherein the function preferably is a power and most preferably a square function, The common idea behind the alternatives for calculating a ST-segment slope value in an ECG signal is to associate to the ST-segment slope value an information that represents the amount of increase of the measured values in the ST-segment with time, or, in other words, the steepness or the slope of ST-segment.

An apparatus according to the invention derives at least one ST-segment slope deviation value information from information of at least two ST-segments slopes, respectively, from a heart of a human or animal, the ST-segment slopes having occurred and preferably been measured at different times. The ST-segment slopes can be derived from the same heart but from different locations. Preferably, this is done with measured data from an individual human or animal, preferably during a defined period of time or from a defined number of heart beats.

This kind of deviation is called first order deviation, and the corresponding values are mentioned as first order ST-segment slope deviation values. They might have further properties going beyond their property of being a ST-segment slope deviation value of the type mentioned above which means that further types of ST-segment slope deviation value can be calculated from ST-segment slope deviation values as a basis. The different types of deviation values are assessment data. The assessment data are thus all based on deviations between at least two ST-segment slopes. These deviations contain a correlation to the arrhythmias of a heart or a part of it.

A “deviation” in this context is assigned to the phenomenon that at least parts of the information of the ST-segment slopes change between measurements of ST-segment slope originating from different heart beats. The deviation is obtained as a result of a certain type of comparison of at lest two ST-segment slope values. Other types of comparison with a goal different from obtaining a deviation value can also be carried out which are described below. A deviation may be a a mathematical difference, but there are also other possibilities. For example, a division can be used, which delivers a quotient which carries the the deviation of the compared ST-segment values in on single value. Alternatively or additionally, a lookup table which, for example, attributes a deviation to a pair of ST-segment slope values, or another known method for deriving a deviation might be used.

If more than two ST-segment slope values from different heartbeats are used for derivation of one or more ST-segment slope deviation values, also statistical methods can be used. Statistical methods help to enhance the quality of obtained information which is present basically in the differences between ST-segment slopes of different heart beats. A deviation can be calculated as a statistical dispersion, for example as a statistical standard deviation by using a standard deviation calculation algorithm as known in statistics or an equivalent deviation calculation method known from mathematics or statistics, for example calculation of a variance or an expectancy value or values describing a frequency distribution. Another possibility is to calculate a discrete derivative with respect to time or number of the heartbeat in a heartbeat sequence in order to obtain the relevant information which is present in the changes between single ST-segment slope of different heart beats. For example, a mean value a median, an expected value or an mathematical average value or another value that represents a value of typical order of magnitude of the derivative can be defined as a ST-segment slope deviation value. Other possibilities are to define frequently appearing differences or a difference between a minimum and a maximum or other representative value as a ST-segment slope deviation value, respectively.

By a statistical calculation the information quality of a ST-segment slope deviation value of any type, or of a characteristic value or a line average value can be enhanced. The statistical method can be applied to first order deviation ST-segment slope deviation values and/or second order local and/or time ST-segment slope deviation values which may be normalized or not normalized which is described below.

The ST-segment slope deviation values or normalized ST-segment slope deviation values can be from the same location of the heart or from a region of the heart or from the whole heart. For example, different electrodes can be used for different locations of the heart. The information of different electrodes or lines can be united in single values by calculation, especially, but not exclusively, by statistics. Information on a greater region of which typical electrodes pick up information mainly can be obtained by using a greater electrode than a typical electrode. It is possible to use an electrode that cover a significant part of the heart or a main part of the heart or the whole heart.

In order to unite information of more than one electrodes, a line average value can be calculated. This can be done using statistics. The line average value does not have to be, but can be, a mathematical average value, A line average value can be a mean value, a median, an expected value or an mathematical average value or another value that represents a value of typical order of magnitude of relevant values.

Into the apparatus, functions of an electrocardiograph can be integrated. The apparatus can also be independent from an electrocardiograph and can obtain the ST-segment slope deviation value from ST-segment slope information which is supplied to the apparatus.

Preferably, the ST-segment slope information is measured with a digital result.

Measurement is not necessarily a part of the data processing device. Input data for the data processing device can also be supplied by a separate measurement unit or be taken from a data memory.

Preferably, the apparatus provides the ST-segment slope deviation value for assessment of the condition. This provision can for example be to a user, to an automated assessment apparatus or to a memory from where it can be retrieved later.

In embodiment of the data processing apparatus, the ST-segment slope information for obtaining the ST-segment slope deviation values has a resolution of less than 5 ms, or 285 μs, preferably less than 100 μs and most preferable less than 50 μs in time and of less than 50 mV or 16 μV in amplitude, preferably less than 1 μV and most preferable less than 1nV. The resolution values are meant to be effective resolution values which might also be achieved by using a worse real resolution and more data, whereof the relevant information can be retrieved by mathematical or statistical methods.

With the proposed amplitude resolution of the input data for the data processing apparatus of 50 mV and a time resolution of 5 ms, some useful results can be obtained. With an amplitude resolution of 16 μV or better and/or a time resolution of 285 μs or better, the deviations correlating with a myocardium condition can be detected in a better quality. The better the resolution, the more precise assessment data result. With an amplitude resolution of 1 μV or better, good results can be achieved. With a resolution of 1nV or better, very good results can be achieved. Also, with a time resolution of 100 μs or better good results can be achieved, and with a time resolution of 50 μs or better, very good results can be achieved. It can, however, be sufficient that only parts of the information that are used for obtaining ST-segment slopes have sufficient resolution. In case that an analog-to-digital-converter is used for digitalisation of input data, it can have a relative resolution that enables the above mentioned absolute resolution at least for relevant ST-segment slope information. An amplifier can be used to adapt the measurement range and the absolute resolution of an analog-to-digital-converter. A person skilled in the art can easily calculate the relative resolution and the number of bits (bit-width) of an analog-digital converter for the measurement from the measurement range and the absolute resolution. The amplitude of the ST-segment preferably fits into the measurement range. At least, the relevant parts of the ST-segment fit into the measurement range. The QRS-complex does not necessarily have to fit in the measurement range completely for one example embodiment of this invention.

In a further embodiment, ST-segment slope deviation values generated by the ST-segment slope can be provided and used for assessment of the level of metabolism activity of the myocardium. The detection of the beginning and the end of ST-segment, respectively, can be made according to standard methods of signal processing.

In a further embodiment, the ST-segment slope deviation value is a function parameter of a function that approximates or matches the ST-segment. It is also possible to find an approximation function for ST-segments. For this, the waveform of the ST-segment is preferably measured by digitising a number of discrete points of the waveform as is for example known from signal theory. The function can, preferably for T-wave, be a wavelet function, one or more spline functions which can be connected or a polynomial function or another function or mathematical relation between time and a T-wave signal that a person skilled in signal processing or mathematics would consider to use and that is preferably based on the measured points of the waveform of ST-segment slope. In this embodiment, the apparatus is configured to approximate ST-segment slope information with a mathematical function. The ST-segment slope can for example be approximated by a linear function, preferably by a segment of a linear function of which more preferably also the end points match with the endpoints of ST-segment.

In a further embodiment, the data processing apparatus can be configured to extract assessment data from ST-segment slope information exclusively. In this embodiment, information from other parts of a PQRST-complex are not used for generating assessment data.

In a further embodiment, the data processing apparatus is configured to calculate a normalized ST-segment slope deviation value by relating the ST-segment slope deviation value which is based on ST-segment slope information to a characteristic ST-segment slope value which is preferably taken from the same source as ST-segment slopes of which the ST-segment slope deviation value has been derived. It is also possible to calculate a characteristic ST-segment slope value from more than one line. This is for example preferred in case that a normalized type of line average value is calculated. The lines are preferably the same ones from which also the line average value is calculated.

The term “source” means in this context that ST-segment slope information is taken from a certain place of measurement on same individual, such as from a certain electrode on a certain place on an individual. The absolute amount of deviations which are the main information carrier for the myocardial metabolism usually changes between different sources of a single individual and especially between different individuals though the contained information is the same. The method of this embodiment has the advantage to render the information of the ST-segment slope deviation values comparable between the sources from one individual as well as between different individuals. This makes it possible to establish standard thresholds for diagnosis of arrhythmias of the heart or a part of it, which can be valid for all or many individual humans or animals of a certain species.

The term “relating” can for example mean a mathematical division or a comparable method known from mathematics or statistics. Alternatively or additionally, a lookup table can be used to associate a normalized ST-segment slope deviation value to the ST-segment slope deviation value and the ST-segment slope characteristic value. The latter method renders the relation process better adaptable, especially in case that the magnitude of the the ST-segment slope value that is to be related to obtain a normalized value and the corresponding characteristic ST-segment slope value do not vary with it each other proportionally.

The idea behind the “relating” is to set two different information types, namely a difference of ST-segment slope value and a characteristic ST-segment slope value in association to each other. This shall result in a new information type in which the information types are mixed in such a way that the influence of the special conditions that are present in one certain individual and that are less or not significant for the intended assessment or diagnosis, is reduced in its presence in the result of the relating process. In certain cases, the relation can, in addition or alternatively to the above mentioned mathematics, be realized as a statistical function or a multiplication.

A “characteristic value” can be a mean value, a median, an expected value or an mathematical average value or another value that represents a value of typical order of magnitude of ST-segment slope values which may be calculated by methods known in mathematics or statistics. It also can mean another value which is typical for the ST-segment slope deviation value in the examined individual, for example a ST-segment slope deviation value that is measured frequently or is a central value of a range of ST-segment slope deviation values which are measured frequently. It is also possible to use this principle of relating a deviation value to a characteristic value for other applications as is discussed below. This relates to a characteristic value ST-segment slope.

It is proposed to consider the myocardium as a regulated part of the body and to assess a state of a regulated process of it. Regulated means that the myocardium has a feedback loop in order to stabilise certain body process characteristics. Such a regulation usually does not lead to a constant process but produces deviations in dependency of the regulation mechanism. One such myocardium process can be a metabolism of a myocardium.

A data processing apparatus for detecting a regulation state of the myocardium process is proposed which can measure a certain characteristic of the myocardium process more than one time and to derive a deviation value from at least two measurements of a signal of the body function. The deviation can be measured and processed to obtain a deviation value. As a myocardium needs stability in most of the processes he carries out, by a deviation value a state of the process of the myocardium can be assessed. A great deviation value means that the regulation process causes great fluctuations of the characteristic which in many cases means a pathological condition of the myocardium.

Information about the myocardium process can be gained by obtaining measurement data from a measurable characteristic which is associated with the myocardium process and which is present in a corresponding signal. To this end, the data processing apparatus comprises a measurement device with at least one measurement path for the flow of information from the ST-segment. For example, the measurement path can comprise a measurement data production unit such as an analog-to-digital-converter for generation of amplitude data and/or time data. If more than one measurement path is used, the measurement paths can be switched to the common measurement data production unit. Also, it is possible that two or more measurement paths have its own measurement data production unit. The measurement data are preferably measured at known times. Subsequent measurement times preferably have substantially constant time intervals between each other.

Preferably, deviations are measured during a short term measurement period in which raw information is acquired. A short term measurement period means that the deviations are measured in a time period of less than one day and preferably less than 15 minutes, especially during at least 1 second, preferably at least 2, 3 or 4 seconds, more preferably at least 30 seconds and most preferably about 200 seconds. In order to derive second order time deviations, a second short term measurement can be carried out at a later point in time. Between the point in time of the first short term measurement and the second short term measurement, a long-term measurement period elapses. Preferably, the long-term measurement period is longer than the short term measurement period. More preferably, the long-term measurement period is longer than 3 hours which can be applied for acute cases or at least one day for less acute cases or at least one week for non-acute cases but for higher precision of event prediction. The long-term measurement period is used to have a time distance in order to derive a second order time deviation value. Preferably, a high relative resolution of for example at least 5·10⁻⁴ is used for the measurement wherein the measurement range is defined as values between a minimum a maximum value of the signal or a relevant part of it.

The deviation value can for example be a difference, especially between two ST-segment slopes that have been measured one after the other in time or can be a standard deviation of measured ST-segment slopes or characteristics of the signal or another value derived from a difference between recurring signals of segments of a signal according to a method that is known in mathematics or statistics.

Preferably, a normalization of the variation is carried out. The purpose of this is to adapt the amount of variation to the myocardium process from which the measurement data are measured such that it can be compared with values of other individuals. In many cases, the measured amount of deviation, the deviation value is derived from an ST-segment slope as described above. The assessment data and/or a diagnosis can be normalized by relating the deviation value to a characteristic value as has been discussed in regard of the myocardial condition. normalized deviation values which are calculated in this way can originate from different measurements at different places or times from the same person or from different persons, wherein the measurements carry information from the same body process. Such normalized deviation values are comparable between different locations of measurement at one person and between measurements at different times. Further, a normalized deviation value from a body can be compared with a normalized deviation value from another body which can have different absolute values. Calculations are preferably carried out by digital data processing.

A data processing apparatus for providing data for assessment of a status of a myocardium process of a body of a human or an animal or a part of it is proposed as an optional embodiment. The apparatus comprises a measurement device which is arranged to measure a characteristic of a process of the myocardium with at least one measurement path for information which is retrieved from the myocardium, and to produce measurement data of the at least one measurement path. Further, the apparatus comprises a calculation device which is arranged to calculate a deviation value from the at least one measurement path. The apparatus can further comprise a normalization device that provides a normalized deviation value for each measurement path which each corresponds to the deviation value of the measurement path related to a characteristic value of the measurement data. The measurement data used for calculation of the characteristic value are preferably from the same measurement path. The characteristic value then belongs to this measurement channel. It also can be a characteristic value that is calculated from measurement data that originate from different measurement paths. Then, the characteristic value is a more general characteristic value which can for example be used for relating it to a line average value of measurement data of some or preferably all of the different measurement paths used for calculation of the more general characteristic value. Such a line average value is calculated from measurement data acquired in a shorter period of time than the measurement data for calculation of the characteristic value. Preferably, the line average value is calculated from measurement values that have been acquired from one single acquisition from each of the relevant different measurement paths. By relating a line average value to a more general characteristic value of, a normalized line average value is calculated. Further, the apparatus can comprise a threshold comparison device which is configured to compare a deviation value or a normalized deviation value or an line average value or a normalized line average value to a threshold value. Such a threshold can for example be obtained by experiments in which common methods for diagnosis for heart conditions or diseases are compared with the corresponding values according to the diagnosis methods based on the data processing apparatus or a corresponding data processing method as disclosed in this patent application. Additionally or alternatively, the threshold comparison device can be configured to compare deviation values or normalized deviation values or line average values or normalized line average values of the same type, but measured at different times or different locations, to each other. Particularly, this can be useful to track a condition or a disease of a person. Additionally or alternatively, the threshold comparison device can be configured to calculate a quotient of two deviation values or normalized deviation values from different measurement paths. The quotient can be compared to a threshold. Single or groups of devices of the apparatus can be arranged in different units which preferably can be locally separated from each other. One or more of the processes carried out by the devices of the apparatus can form a method for providing concerning myocardium or for assessing the condition of the myocardium.

In a further embodiment, the apparatus is configured to interrelate a ST-segment slope deviation value or a normalized ST-segment slope deviation value of a first source of an individual with a ST-segment slope deviation value or a normalized ST-segment slope deviation value, respectively, of a second source of the same individual and to generate a second order local ST-segment slope deviation value. A source can be an electrode which is placed on the body of the person or the animal to preferably measure a certain part of the heart or a certain part more than other parts. Also, an electrode or a line which is connected to an electrode can be considered as a source. It is also thinkable that a source can be an electric or electromagnetic field from the heart which are preferably received from a certain part of the heart by a suitable receiver. The first and the second source yield information from different locations at the heart.

An “interrelation” in the context of calculating a certain type of ST-segment slope value means to calculate a second order ST-segment value that represents a local difference of ST-segment deviation values at different locations or regions at the heart, wherein one of the different locations or regions can comprise the other one. The myocardial conditions of different locations at the heart can vary. This can be correlated to certain types of arrhythmia. The local difference can also be present between a particular location in comparison to the average condition of the whole heart or a part without this particular location. In order to calculate values that represent this correlation, values that represent the certain location or the whole heart or the whole heart without the particular location can be calculated. With these values, the interrelation can be carried out. The interrelation can be a mathematical subtraction or another method that is described in regard of calculation a deviation value. Further possibilities are described above in view of calculating a deviation value and can also be used for interrelating values from different sources.

In order to enhance the significance of the obtained second order local ST-segment slope value, statistical methods for uniting the information in this value can be applied, preferably the methods described in regard of obtaining a deviation value. The ST-segment slope values of the deviation calculation correspond to ST-segment slope deviation values in case of of calculating a second order local ST-segment slope value. It is also possible to calculate a a second order local ST-segment slope value from a plurality of ST-segment slope values in applying an appropriate calculation of the temporal deviations and the local differences.

For example, second order local deviation values can be obtained by calculation of differences between information from different locations. ST-segment deviation values on which this calculation is based are derived from ST-segment slopes in a first step are called first order deviation values in this context. They are preferably normalized deviation values as described above. So, second order deviation values can be obtained from differences between first order deviation values regarding location as described above or regarding time as described below. In order to obtain one second order local deviation values, four measurements in single measurement paths comprising a single electrode each can be carried out. At least two of these measurements can be carried out using the first source and at least two measurements can be carried out using the second source. Also the apparatus is configured to calculate a line average value of ST-segment slope deviation value or a normalized ST-segment slope deviation value from a few and/or all sources representing different locations of the heart.

As mainly the leads V3, V4, V5, V6 or V3, V4, V5, V6, V7, V8, V9 belong to the left ventricle, V1, V2 or V1, V2, V3R, V4R, V5R, V6R belong to the right ventricle, these leads are preferably involved in generating a local deviation value or a line average value. Interrelating different leads is advantageous, because local differences can be recognised which are a hint to a myocardial metabolism disorder. The myocardium can have a locally deviating metabolism which can go along with a probability of an ectopic focus in myocardium. The locations of the electrodes can be the standard locations.

There might be cases in which normalization is not necessary for comparison between different sources of an individual, for example in individuals which have very similar ST-segment slopes from different sources. However, this is not the normal case, but still, it is possible. For example, the leads V1, V2, V3, V4, V5, V6 and/or V1, V2, V3, V4, V5, V6, V7, V8, V9 and/or V1, V2, V3R, V4R, V5R, V6R can be compared one with another to generate subtraction values or to generate relation values or calculate the line average value, for example values that represent a quotient.

In general, it is not required to use leads V1 to V6 according to Wilson. It is also possible, with less local resolution, to use the leads aVR, aVL and aVF according to Goldberger. It is also possible to use further known arrangements of electrodes and leads.

In case of using the unipolar leads according to Goldberger, it is preferred to carry out measurements between different combinations of the leads. Then, quotients or differences of two of the results can be calculated, respectively. A comparison between the quotients or differences can be interpreted as a second order local deviation value. With a second order local deviation value can be assessed whether there are local differences of a disease in the myocardium. This can also be done with the standard Einthoven electrode and lead arrangement with leads I, II and

In a further embodiment, the data processing apparatus is configured to process a number of at least 2 ST-segment slopes, preferably at least 60 ST-segment slopes and most preferably about 200 ST-segment slopes and/or to process a number of ST-segment slopes of a PQRST-complex during at least 1 second, preferably at least 2, 3 or 4 seconds, more preferably at least 30 seconds and most preferably about 200 seconds to obtain normalized ST-segment slope deviation values or ST-segment slope deviation values and to calculate a second order deviation value of the obtained normalized ST-segment slope deviation values or ST-segment slope deviation values. As at least three measurements are necessary to carry out a second order deviation, at least three heartbeats are acquired which can be acquired in one second at very fast heart rate and in 2, 3 or 4 seconds with slower heart rates. With a number of at least measured 60 ST-segment slopes, the quality of the data produced by the data processing apparatus can be improved significantly by better statistics. A good trade-off between measurement time and high-quality is acquiring about 200 ST-segment slopes which corresponds to measurement time of typically 200 seconds with a sitting patient which has quite slow heart rate. For even better quality, longer measurements times can be used. The first order deviation information is contained in the ST-segment slope deviation values or normalized ST-segment slope deviation values, wherein the second order deviation information is contained in differences between the normalized ST-segment slope deviation values or ST-segment slope deviation values from different sources or measured at different times.

In a further embodiment, the data processing apparatus can be configured to compare a ST-segment slope deviation value or a normalized ST-segment slope deviation measured at a first time to a ST-segment slope deviation value or a normalized ST-segment slope deviation value, respectively, measured at a second time, wherein the second time is different from the first time, and to generate a time deviation value. As these results in a second order deviation regarding time, at least three measurements of ST-segment slope are required. A first difference between a first combination of two of the ST-segment slopes delivers a first order deviation value and a second difference between a second combination of the ST-segment slopes delivers a second to first order deviation value. The first and the second to first order deviation value can be compared to obtain their second order time deviation value. Preferably, the values are obtained as normalized deviation value, wherein the normalized deviation values are calculated from deviation values by division by a characteristic value as described above. It is also possible to divide a second order time deviation value which is based on non-normalized first order deviation values by a characteristic value. By such a second order time deviation value, a trend of the ST-segment slope deviation value or the normalized ST-segment slope deviation value can be represented. When a comparison between individuals is required, the normalized values are preferred. With such a trend, an event time period or an event point in time can be extrapolated after which or at which, respectively, it is expected that the ST-segment slope deviation value or the normalized ST-segment slope deviation value reaches a predefined value, for example a threshold value. Extrapolation methods are well known in mathematics, information technology and engineering. In this way, a prognosis can be made when a certain condition of the heart can be expected. For example, an event time period can be calculated after which an atrial flutter, atrial fibrillation, sick sinus syndrome or a sino atrial node dysfunction, atrioventricular heart block, ventricular premature beats, polymorphic ventricular premature beats, ventricular tachycardia episodes, ventricular fibrillation, sinus extrasystole, atrial extrasystole, atrioventricular extrasystole, supraventricular paroxysmal tachycardia, arrhythmogenic right ventricular cardiomyopathy can be expected. The conditions mentioned above can be derived from ST-segment slope information as described in this patent application.

The proposed measurement of a second order deviation value preferably takes at least 1 to four seconds, preferably at least 30 seconds of overall measurement time which provides better data quality and more preferred about 2 to 3 minutes of overall measurement time which provides an optimum trade-off between quality and measurement time. For even better quality, longer measurements times can be used. With such a data processing apparatus, a development of a myocardial metabolism can be represented in a value which is based on ST-segment slope.

Particularly, from the change rate of non-normalized or normalized ST-segment slope deviation values or ST-segment slope deviation values, an expected time to an acute myocardial infarction can be calculated. The calculation can be based on a lookup table or a function which is based on a typical correlation between the time to atrial flutter, atrial fibrillation, sick sinus syndrome or ventricular tachycardia episodes or ventricular fibrillation and a normalized ST-segment slope deviation value or ST-segment slope deviation value.

A second order distance in time of the deviation values for calculation of a second order time deviation value or a normalized second order time deviation value is preferably greater than a first order distance in time of the measurement values for the first order deviation values on which the non-normalized or normalized second order time deviation values is based.

The different measurements to derive a second order time deviation value preferably originate from the same location but are measured at different times such that a second order local deviation value or a normalized second order local deviation value can be obtained.

In a further embodiment, the data processing apparatus comprises a measurement device for generating ST-segment slope information from an individual human or animal by measurement, wherein the measurement device preferably has a resolution of less than 5 ms, or less than 285 μs, preferably less than 100 μs and most preferable less than 50 μs in time and/or of less than 50 mVor less than 16 μV in amplitude, preferably less than 1 μV and most preferable less than 1nV. Preferably, in these values also the precision and/or interference into electrodes and/or leads at standard conditions in a hospital is comprised.

The data processing apparatus has preferably one or more features of a common electrocardiograph with improved resolution. Particularly, it can comprise a common number of lines and electrodes. Though measurement with common electrodes is possible, electrodes for the measurement device are preferably low noise electrodes. Also, wireless electrodes are preferred.

In a further embodiment, the data processing apparatus comprises a signalling device which is configured to signalise and/or display a deviation value and/or a normalized ST-segment slope deviation value and/or a diagnosis proposal and/or a diagnosis that is made automatically to a person and/or a risk for a heart condition and/or a heart disease. The diagnosis proposal and/or the diagnosis and/or the risk for a heart condition and/or a heart disease is derived from the measurements and/or ST-segment slope deviation values as explained below. For example, non-normalized or normalized deviation values of amplitude, time period, area and/or function parameters, event time period or event point in time, risk and/or presence and/or extent of: atrial flutter, atrial fibrillation, sick sinus syndrome or a sinoatrial node dysfunction, atrioventricular heart block, ventricular premature beats, polymorphic ventricular premature beats, ventricular tachycardia episodes, ventricular fibrillation, sinus extrasystole, atrial extrasystole, atrioventricular extrasystole, supraventricular paroxysmal tachycardia, arrhythmogenic right ventricular cardiomyopathy can be signalled as a single information or an arbitrary combination. Preferably, the information can be signalled or displayed for each lead separately. The signalling device can be arranged at another location as the data processing apparatus or are measurement device which is connected to the human or animal. The signalling device and the data processing apparatus can for example communicate via a data link or physical memory can be exchanged.

In a further embodiment, the data processing apparatus comprises a data acquisition device comprising a data memory which is configured to record deviation data or normalized deviation data or diagnosis proposal data or automatic diagnosis data and/or a data remote transfer device to transfer deviation data or normalized deviation data or diagnosis proposal data or automatic diagnosis data to a remote device of the data processing apparatus, wherein, preferably, the data acquisition device is locally separable from a signalling device at which data from the data memory can be displayed.

In a further embodiment, a non-normalized or normalized first order or second order ST-segment slope deviation value represents a variable to assess if and/or to which extent a metabolism of the heart and/or arrhythmias of heart or a part of it is active. Non-statistical or statistical ST-segment slope deviation values and Non-statistical or statistical normalized ST-segment slope deviation values as well as second order deviation values can, for example, be displayed by the apparatus.

In a further aspect of the invention, an assessment apparatus for an assessment of a condition of at least a part of a myocardium or the whole myocardium of a heart of an individual human or animal and/or a heart disease is proposed. This can also comprise assessing an event time period or an event point in time as described above. The assessment apparatus can use a ST-segment slope deviation value or/and a normalized ST-segment slope deviation value or/and a second order local deviation value or/and a second order time deviation value which have been obtained by a data processing apparatus as described above. A. Data provided by the data processing apparatus are considered assessment data. The assessment apparatus can be integrated into the data processing apparatus. Additionally or alternatively, assessment data can be supplied by data transfer from the data processing apparatus or by reading out a memory which has been recorded by a data processing apparatus.

In an embodiment, the assessment apparatus comprises a data processing apparatus according to one of the embodiments described in the present patent application. This does not necessarily mean that the assessment function is executed in the same location as the data processing, but it can also be the case. Alternatively, the assessment can be done locally separated from the data processing and/or the data acquisition from the human or animal. A centralized assessment can be used for assessment of assessment data from a plurality of data processing apparatuses. It is also possible to have the data processing apparatus and the assessment apparatus at the same location and to process and assess acquired data from a plurality of humans or animals which have been acquired at another location as the location of the data processing apparatus and the assessment apparatus.

In at least the following paragraphs up to the table of figures, the mentioned values can be non-statistical or statistical values.

In an embodiment of the assessment apparatus, the assessment apparatus is configured to assess

A) risk and/or presence and/or extent of sick sinus syndrome and/or sinoatrial node dysfunction and/or atrioventricular heart block

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few or all sources representing different locations and/or a big part of the heart, and/or

B) risk and/or presence and/or extent of sinus extrasystole and/or atrial extrasystole and/or atrioventricular extrasystole

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few or all sources representing different locations and/or a big part of the heart, and/or

C) risk and/or presence and/or extent of supraventricular paroxysmal tachycardia

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few or all sources representing different locations and/or a big part of the heart, and/or

D) risk and/or presence and/or extent of atrial flutter

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few or all sources representing different locations and/or a big part of the heart, and/or

E) risk and/or presence and/or extent of paroxysmal atrial fibrillation

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few or all sources representing different locations and/or a big part of the heart, and/or

F) risk and/or presence and/or extent of atrial fibrillation

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few or all sources representing different locations and/or a big part of the heart, and/or

G) risk and/or presence and/or extent of ventricular tachycardia episodes

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few sources representing different locations and/or a big part of the heart, and/or

H) risk of ventricular fibrillation

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few sources representing different locations and/or a big part of the heart, and/or

I) risk of sudden cardiac death

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few or all sources representing different locations and/or a big part of the heart, and/or

K) risk and/or presence and/or extent of ventricular premature beats and/or polymorphic ventricular premature beats

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few sources representing different locations and/or a big part of the heart, and/or

L) risk and/or presence and/or extent of arrhythmogenic right ventricular cardiomyopathy or dysplasia and its complications

by a ST-segment slope deviation value and/or normalized ST-segment slope deviation value of one or a few sources representing different locations of the right ventricle of the heart and/or by an average value of ST-segment slope deviation values and/or an average value of normalized ST-segment slope deviation value of a one or a few sources representing different locations of the right ventricle of the heart, and/or

M) an event time period or an event point in time for arrhythmias of a heart or a part of it as mentioned in items A) to L), N) and O)

by a second order time deviation value or normalized second order time deviation value of a ST-segment slope as mentioned under A) to M),
in respect of certain condition or disease,
particularly an event time period to an acute myocardial infarction or an event point in time of an arrhythmias of a heart or a part of it
by a speed of decreasing or increasing of second order of ST-segment slope deviation value or second of normalized ST-segment slope deviation value in time and/or an average value of ST-segment slope deviation value or an average value of normalized ST-segment deviation value, and/or

N) risk and/or presence and/or extent of right and left bundle branch block

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few sources representing different locations and/or a big part of the heart, and/or

O) risk and/or presence and/or extent of arrhythmogenic ectopic focus in myocardium

by a ST-segment slope deviation value and/or normalized ST-segment slope deviation value of one source representing different locations and/or a big part of the heart and/or by an average value of ST-segment slope deviation values and/or an average value of normalized ST-segment slope deviation value of a few sources representing different locations and/or a big part of the heart, and/or

P) a location of the arrhythmias of a heart or a part of it as mentioned as item A) to M) by a second order local deviation value or a normalized second order local deviation value of ST-segment slope under A) to M) for the arrhythmias of a heart or a part of it, respectively.

At the assessment of the condition relates a continuous value which can be assessment information and which by itself does not have any threshold to arrhythmias of a heart or a part of it, it is possible to assess the extent of arrhythmias of a heart or a part of it. Therefore, also conditions can be recognised which not yet have expressed themselves as disease. In this way, a risk for the disease or worse arrhythmias can be assessed. In contrast, diagnose of arrhythmias is made by comparison of assessment information with a threshold. An assessment can comprise providing assessment data in a context of certain arrhythmia which is correlated to the assessment information. An assessment can also comprise finding one or more arrhythmias of a heart or a part of it on basis of single assessment information which can be a first order or second order deviation value or an average value of deviation values, or a combination of such assessment information. The assessment of arrhythmias of a heart or a part of it can additionally be made dependent on the situation or condition in which a patient is.

For example, if patient comes to a doctor or a hospital with arrhythmia, it can be distinguished whether the different types of arrhythmias can cause more severe or fatal arrhythmias. Sometimes it is difficult to find out about the presence of paroxysmal arrhythmias, which cannot be detected by usual diagnostic methods such as rest electrocardiography or 24 or more hours of electrocardiography monitoring, and to assess quantitatively the risk of fatal arrhythmias. By using a method as described in the present patent application, the presence or risk or extent of different types of arrhythmias can be quantitatively detected.

In another example, a person comes for a routine control without any symptoms. Then, the presence of arrhythmias or a risk of arrhythmias can be detected.

In a practical example, different types of arrhythmias can be recognised by a statistical normalized ST-segment slope deviation value. As a statistical method, a standard deviation calculation can be carried out to obtain the ST-segment slope deviation value for normalization. To obtain the characteristic value for normalization, a mean value can be calculated of the same ST-segment slope values that are used for obtaining the ST-segment slope deviation value for normalization. Normalization is carried out using a division as the process of relating.

In this practical example, the calculation can be done with

all of the leads V1, V2, V3, V4, V5, V6 and/or
leads V1, V2, V3, V4, V5, V6, V7, V8, V9 and/or
standard leads I, II, III and/or
leads aVR, aVF, aVL.

A statistical normalized ST-segment slope deviation value calculated according to this practical example that ranges

| | |
|---|---|
| from 0.00 up | sick sinus syndrome and/or sinoatrial node |
| to 0.04 means | dysfunction an/or atrioventricular heart block, |
| from 0.04 up | normal, |
| to 0.15 means | |
| from 0.15 up | low risk of supraventricular tachycardia or sinus |
| to 0.2 means | extrasystole or atrial extrasystole, |
| from 0.21 up | high risk or presence of supraventricular |
| to 0.25 means | tachycardia or sinus extrasystole or atrial |
| | extrasystole and low risk of presence of atrial |
| | flutter or paroxysmal atrial fibrillation, |
| from 0.26 up | high risk or presence of atrial flutter or |
| to 0.31 means | paroxysmal atrial fibrillation, |
| from 0.32 upt | presence of atrial fibrillation and high risk or |
| to 0.45 means | episodes of ventricular tachycardia, |
| from 0.45 or | high risk of ventricular tachycardia and ventricular |
| greater means | fibrillation and high risk of sudden cardiac death. |

It is proposed to assess, determine or diagnose a type of condition or disease from the value of a variable representing a non-statistical or statistical, non-normalized or normalized ST-segment slope deviation value. An assessment or a diagnosis can be made on basis of one single measurement session, particularly without taking into account a further measurement that is made not during the time period of this measurement session.

A gradual decrease of a non-statistical or statistical, non-normalized or normalized ST-segment slope deviation value to the half or less in specified time (for example 2, 4, 6, 12 or 24 hours or more) of at least one average value of normalized deviation value of ST-segment slope from at least one of leads V1, V2, V3, V4, V5, V6 and/or at least one of V1, V2, V3, V4, V5, V6, V7, V8, V9 and/or standard leads I, II, III and/or leads aVR, aVF, aVL from normal values to lower values (for example, from value 0.04 to value 0.02) means the risk and/or presence and/or extent, depending on the individual, of sick sinus syndrome and/or sinoatrial node dysfunction and/or atrioventricular heart block. The more significant the decrease of average value of normalized deviation value of ST-segment slope from leads V1, V2, V3, V4, V5, V6 and/or V1, V2, V3, V4, V5, V6, V7, V8, V9 and/or standard leads I, II, III and/or leads aVR, aVF, aVL is, the more severe is the extent of sick sinus syndrome and/or sinoatrial node dysfunction and/or atrioventricular heart block.

An increase of two times or more of a first order ST-segment slope deviation value of at least one of the leads V3R, V4R, V5R, V6R, V1, V2, V3, V4, V5, V6, V7, V8, V9, the standard leads I, II, III, and/or leads aVR, aVF, aVL in interrelation with a line average value of normalized deviation value of ST-segment slope of other leads means high risk and/or presence of ventricular premature beats and/or polymorphic ventricular premature beats. The more significant the increase of the deviation value of normalized ST-segment slope of at least one of V3R, V4R, V5R, V6R, V1, V2, V3, V4, V5, V6, V7, V8, V9 leads, I, II, III standard leads, aVR, aVF, aVL leads in interrelation with the average value of normalized deviation value of ST-segment slope of other leads is, the more severe is the extent of ventricular premature beats and/or polymorphic ventricular premature beats. If the increased normalized deviation value of ST-segment slope is in one or a few of V3R, V4R, V5R, V6R, V1, V2 leads, the risk and/or presence of ventricular premature beats are expected to be from right ventricle. If the increased normalized deviation, value of ST-segment slope is in at least one of V3, V4, V5, V6, V7, V8, V9 leads, the risk and/or presence of ventricular premature beats would be from left ventricle.

The decrease to the half or less of the normalized deviation value of ST-segment slope of one or a few of V4, V5, V6, V7, V8, V9 leads in interrelation with the average value of normalized deviation value of ST-segment slope of other leads is high risk and/or presence of left bundle branch block. The more significant is the decrease of the normalized deviation value of ST-segment slope of one or a few of V3, V4, V5, V6, V7, V8, V9 leads in comparison with the average value of normalized deviation value of ST-segment slope of other leads, the more severe is the extent of left bundle branch block.

The decrease to the half or less of the normalized deviation value of ST-segment slope of one or more of V3R, V4R, V5R, V6R, V1, V2, V3 in interrelation with the average value of normalized deviation values of ST-segment slopes of other leads is risk and/or presence of right bundle branch block. The more significant is the decrease of the normalized deviation value of ST-segment slope of one or a few of V3R, V4R, V5R, V6R, V1, V2, V3 leads in comparison with the average value of normalized deviation value of ST-segment slope of other leads, the more severe is the extent of right bundle branch block.

The two times increase or more of the normalized deviation value of ST-segment slope of one or a few of V3R, V4R, V5R, V6R, V1, V2, V3 in comparison with the average value of normalized deviation values of ST-segment slopes of other leads is risk and/or presence of arrhythmogenic right ventricular cardiomyopathy or dysplasia. The more significant is the increase of the normalized deviation value of ST-segment slope of one or a few of V3R, V4R, V5R, V6R, V1, V2, V3 leads in comparison with the average value of normalized deviation value of ST-segment slope of other leads, the more severe is the extent of arrhythmogenic right ventricular cardiomyopathy or dysplasia and it's complications. It also can be useful for detecting the risk and/or presence of Brugada syndrome and its complications.

An assessment apparatus is proposed which comprises an assessment signaling device for an assessment of arrhythmias of a heart or a part of it according to at least one of the items A) to M) of claim 16 to a person or to transfer corresponding data to another unit as the data processing apparatus.

Preferably, a a certain sequence of calculations is applied. Not all of the instances of sequence have to be carried out in any case. First, a ST-segment data set is extracted from raw ECG data which is known from the state of the art. Then, a ST-segment slope value is calculated from the ST-segment data. This is done with at least two ST-segment data sets, such that least two ST-segment slope values are obtained. Then, a deviation value is derived from at least two ST-segment slopes. The useful information is contained in the differences between the single ST-segment slope values. To calculate the derivation value, a mathematical difference can be calculated, but this is not the only possibility. Also, other functions that combine to values to obtain a third value in a reproducable manner, such as a division of a dividend y a divisor, or a logarithm of a value to the basis of the logarithm, or an exponential function of a value and its basis, for example, can be used as a derivation value, as a difference in the two input values is expressed in the result value which is a single value. It is preferred to use the mathematical difference as it is easy to calculate and yields good results.

Having calculated a derivation value, it is an option to calculate a normalized deviation value by use of a characteristic value. The characteristic value represents a typical ST-segment slope value that might be a statistically expected value, an average value, a mean value, a median value or another value that is typical for the deviation value and that is based on a plurality of deviation values.

It is also possible to normalize to a characteristic value of ST-segment slope values before deriving the derivation values. Then, normalized derivation values are obtained by the derivation calculation.

It is proposed that distinct points of the ST-segment can be defined as an initial point and an end point of ST-segment

Preferably, the data processing apparatus is configured to approximate or describe ST-segment slope information with a mathematical function.

It is proposed that a location at the arrhythmias of a heart or a part of it as mentioned as items A) to M) of claim 16 is obtained by a second order local deviation value or a normalized second order ST-segment slope local deviation value under A) to M) of claim 16 for the arrhythmias of a heart or a part of it, respectively.

The normalized ST-segment slope deviation value can also be statistical values. The information can be more clearly present in statistical values.

A second order time deviation value or a second order time deviation value can also be a statistical second order time deviation value or a statistical or a second order time deviation value, respectively.

In a further aspect of the invention diagnosis method is provided, according to which the diagnosis of a condition or a disease of a heart is made by applying an assessment as described above.

### TABLE OF FIGURES

Embodiments of the invention, as an example only, are depicted in the attached figures, in which

FIG. 1 shows a schematic diagram of a measurement of T-wave information from a human heart,

FIG. 2 schematically shows a PQRST complex which can be measured from a human heart,

FIG. 3 schematically shows an ST-segment and features of it which can be used to derive a deviation value,

FIG. 4 shows a schematic diagram of flow of information in a data processing apparatus,

FIG. 5 shows a schematic diagram of flow of information in an assessment apparatus,

FIG. 6 shows a schematic diagram of relationships between a ST-segment slope deviation value and conditions of a myocardium,

FIG. 7 shows a schematic diagram of flow of information from the myocardium to normalized ST-segment slope deviation values which can also be statistical values,

FIG. 8 shows a schematic diagram of flow of information from normalized T-wave deviation values of the myocardium to a second order deviation value, and

FIG. 9 shows a schematic diagram of flow of information from normalized T-wave deviation values the myocardium to a second order time deviation value.

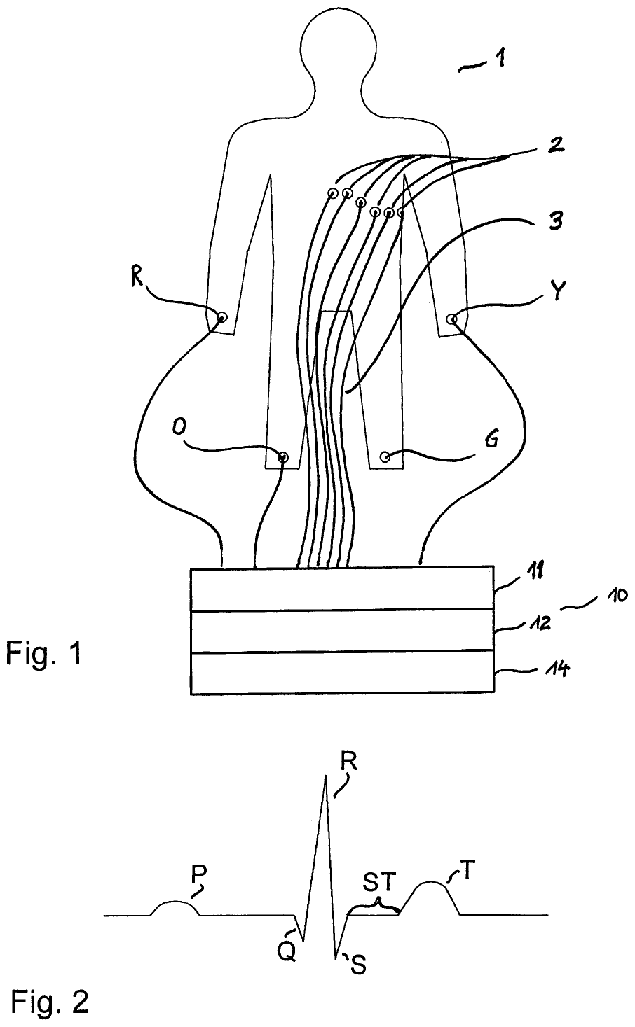

FIG. 1 schematically shows a human 1 in a face-to-face front view. On his upper part of the body, six electrodes 2 are applied for measurement of electric signals produced by the heart of the human 1. The electrodes 2 are connected to a measurement device 11 via cables 3 which together form the well-known leads 1 to V6. Alternatively, wireless electrodes can be used. The measurement device 11 produces ST-segment information from ST-segments in the electric signals. The measurement device 11 is connected to a data processing apparatus 12 and forwards the ST-segment information to it. The data processing apparatus 12 derives deviation values from the ST-segment information. The deviation values can be normalized or non-normalized deviation values and considered as assessment data. They can also be statistical data in which the information is present more expressively. The assessment data are input into an assessment apparatus 14. The assessment apparatus 14 assigns the condition or a disease of a myocardium or part of it or the heart of the human 1 on basis of the assessment in data. The condition or disease can be displayed, transferred to an external unit for further use for example for external recording or alarming a medical staff or service and/or saved in a memory.

In FIG. 1, the reference leads R at the right hand, G at the left hand, Y at the left foot, and 0 at the right foot are shown. As is known in cardiology, a virtual reference point for the leads V1 to V6 from the electrodes 2 can be calculated form signals that are present between the leads R, Y and G with reference to lead 0. It is preferred for more precise results to do so in the present invention, also. The calculation of the reference signals in the torso can be executed as follows:

QVR=R/(Y*G), QVL=Y/(R*G) and QVF=G/(R*Y)

These virtual reference points are located in the torso and thus closer to the heart. It is possible to use a mean value of QVR, QVL and QVF as a reference, also. It is also possible to exchange the leads 0 and G such that G is the reference for the leads R, Y and 0.

FIG. 2 schematically shows a PQRST complex which can be measured electrically from a human heart. The waveform represents a voltage which is plotted in vertical direction over time which is displayed in horizontal direction. A ST-segment is labelled with the reference sign ST. T-wave is labelled with T and appears after it.

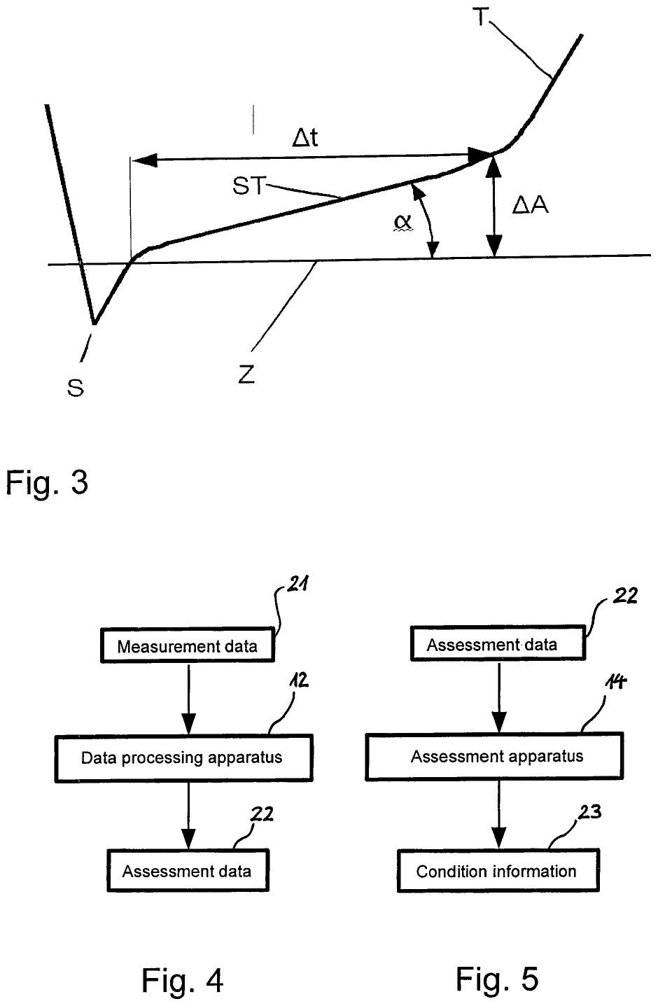

FIG. 3 schematically shows a ST-segment angle α in an ECG voltage signal or waveform shown as ECG diagram ECG from point S in the QRST-complex to the beginning of T-wave. A zeroline Z at the voltage 0V is also shown. ST-segment information is contained in the slope of ST-segment ST.

A ST-segment slope value can be calculated from the slope or the inclination or the gradient in respect to time of ST-segment ST which corresponds to the ST-segment angle α. Especially, the ST-segment slope value can be calculated by a quotient of an amplitude difference ΔA over ST-segment ST divided by a time difference Δt of the time period of the amplitude difference. In FIG. 3, ΔA is depicted over the whole time of ST-segment. It is, however also possible to use only a part of ST-segment ST for calculation of a ST-segment slope value. Particularly, one or more dedicated point in ST-segment ST can be used to define the time period for determination of ST-segment slope, for example at the middle or at a certain fraction of ST-segment or a the beginning and the end. The ST-segment slope value can be calculated as a an average or mean value or expected value or an median amplitude value of at least two single results based on parts of ST-segment, especially if ST-segment ST does not have a constant slope. Also, methods of differential calculus can be used, for example deriving ST-segment ST in respect of time an using this information. For example, a maximum value or some other characteristic value of this data or a statistical value like a mean value or average can be determined as a ST-segment slope value.

FIG. 4 schematically shows a data flow through a data processing apparatus 12. Measurement data 21 which originate from a measurement of T-wave are input into the data processing apparatus 12. The measurement data can also be measurement signals. The data processing apparatus 12 derives first and/or second order deviation values which can be normalized or non-normalized, from the measurement data 21. The deviation data can further be processed to obtain assessment data 22 or to serve as assessment data as such. For example, a skilled person can assess a condition or diagnose a disease by first and/or second order deviation values. For example, current deviation values can be compared with deviation values which have been measured before. Assessment data 22 are output from the data-processing apparatus 12.

FIG. 5 schematically shows a data flow or through an assessment apparatus 14. Assessment data 22 from a data processing apparatus 12 are input into the assessment apparatus 14. The assessment apparatus 14 can compare assessment data with a threshold in order to assess arrhythmia condition of a heart. Corresponding condition information 23 can be output from the assessment apparatus 14 by a display or another signal and/or the condition information can be recorded and/or transferred to an external unit. The condition information can for example be a diagnosis proposal or and automatically made a diagnosis. Such a diagnosis proposal or diagnosis can comprise a condition or disease type, a risk for a condition or disease type, and extent of the present condition or disease, a time period after which a certain event regarding the the condition or disease is expected or a point in time at which this event is expected. Such an event can for example a sudden cardiac death or an arrhythmogenic right ventricular cardiomyopathy.

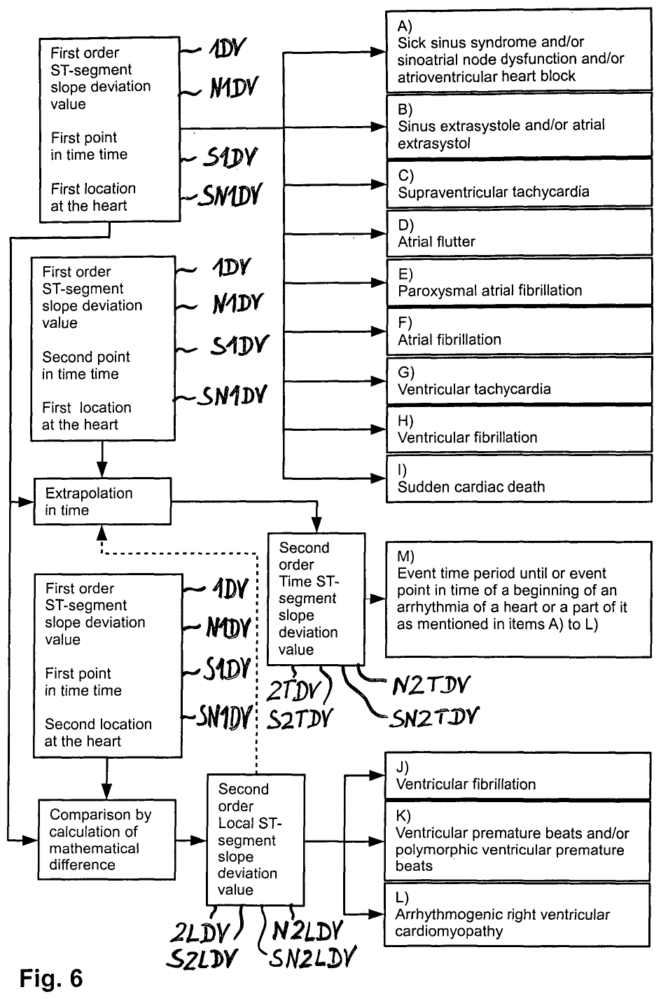

FIG. 6 shows a schematic diagram in which conditions of the heart A) to L) are mentioned and correlated with their respective way of calculation of their risk or presence. Alternatively to using a ST-segment slope deviation value 1DV, 2LDV, 2TDV, also a normalized ST-segment slope deviation value N1DV, N2LDV, N2TDV of one or more sources and/or an statistical value ST-segment slope deviation value S1DV, S2LDV, S2TDV, SN1DV, SN2LDV, SN2TDV such as an average of ST-segment slope deviation value and/or an an statistical value such as an average value of normalized ST-segment slope deviation value representing at least a location at the heart can be used with the specific advantages of these types of values.

The reference numerals contain a S in case that the value is a statistical value. They contain a N in case that the value is a normalized value. The reference numerals contain a 1 in case that the value is a first order deviation value, and a 2 in case that the value is a second order value. They contain a T in case that a second order deviation value is based on a deviation in time. They contain a L in case that a second order deviation value is based on a local deviation.

The conditions A) to I) which are types of arrhythmia are correlated to a first order ST-segment slope deviation value 1DV, N1DV, S1DV, SN1DV. The conditions J) to L) which are further types of arrhythmia are correlated to a second order local ST-segment slope deviation value 2LDV, N2LDV, S2LDV, SN2LDV. The N in the Reference numeral means a normalized type of ST-segment slope deviation value, and the S in the reference numeral means a statistical type of ST-segment slope deviation value. ST-segment slope deviation values can be normalized as well as statistical. Reference numerals without an N or an S mean non-normalized and non statistical types of ST-segment slope deviation values.

The risk or presence of a type of arrhythmia according to A) to L) can be assessed of diagnosed by comparison of the corresponding value with a threshold, which is not explicitly shown in FIG. 6.

The estimation of a point in time or a time period to a certain event regarding arrhythmia or occurrence of a certain condition or certain ST-segment slope value, according to M), requires an extrapolation of the development of ST-segment slope deviation values which is represented by a second order local ST-segment slope deviation value 2TDV, N2TDV, S2TDV, SN2TDV. The point in time or the time period can be calculated or represented by a second order local ST-segment slope deviation value 2TDV, N2TDV, S2TDV, SN2TDV. To calculate a second order local ST-segment slope deviation value 2TDV, N2TDV, S2TDV, SN2TDV, first order ST-segment slope deviation values 1DV, N1DV, S1DV, SN1DV representing at least two different points in time can be used. It is also possible to interpolate a second order local ST-segment slope deviation value 2LDV, N2LDV, S2LDV, SN2LDV in time which can be done in the same way as with a first order ST-segment slope deviation value 1DV, N1DV, S1DV, SN1DV as basis. The flow of information is shown by a dotted line for this case. It also includes calculating at least two second order local ST=segment slope deviation values for different different points in time as the calculation of a second order time ST-segment slope deviation value

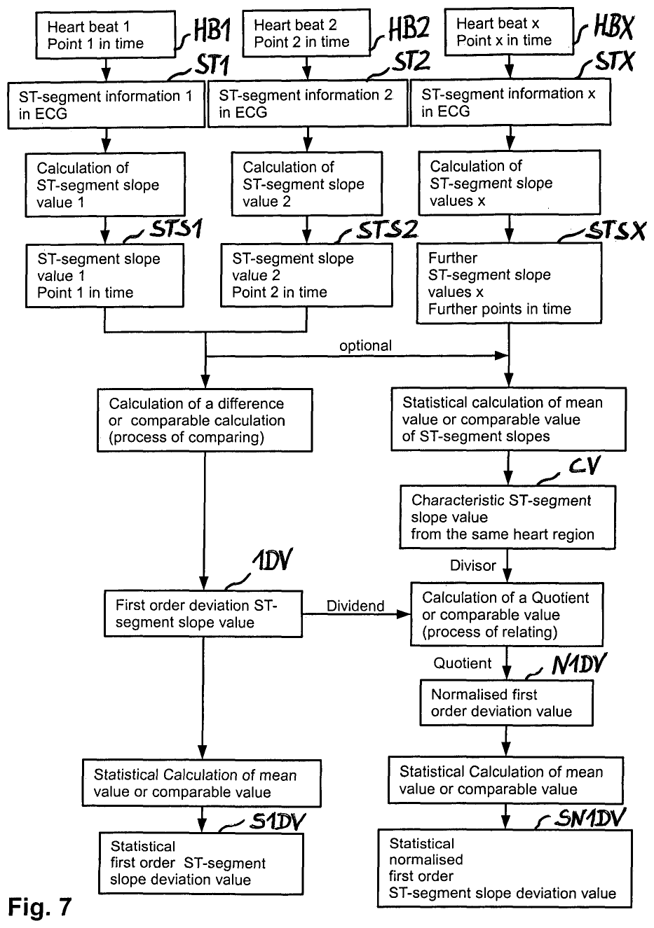

FIG. 7 schematically shows a diagram of information flow from the metabolism of a myocardium which produces electrical signals, up to a statistical ST-segment slope deviation value of first order which optionally can be normalized. The information chain begins with beats HB1, HB2 of a heart of which the condition shall be estimated. Following each other in time, several ST-segments of such heart beats HB1, HB2 are measured, whereof two specific heart beats HB1 and HB2 are mentioned in FIG. 7. A calculation of a normalized ST-segment slope deviation value can be based on additional heart beats HBX.

The measurement of the ST-segment produces ST-segment information ST1 and ST2, respectively. Then, from each ST-segment information 1 and 2, a ST-segment slope value STS1 and STS2, respectively, can be calculated. The result is called a first order deviation value 1DV.

In the next step to a ST-segment slope deviation value 1DV, the ST-segment slope values STS1 and STS2 are interrelated to each other within the meaning of a difference in a general sense. So, one can come from a value that describes the steepness of the slope to a deviation value that describes difference between these steepnesss. The probably simplest way to obtain a ST-segment slope deviation value 1DV is to calculate a mathematical difference between the two ST-segment slope values 1 and 2.

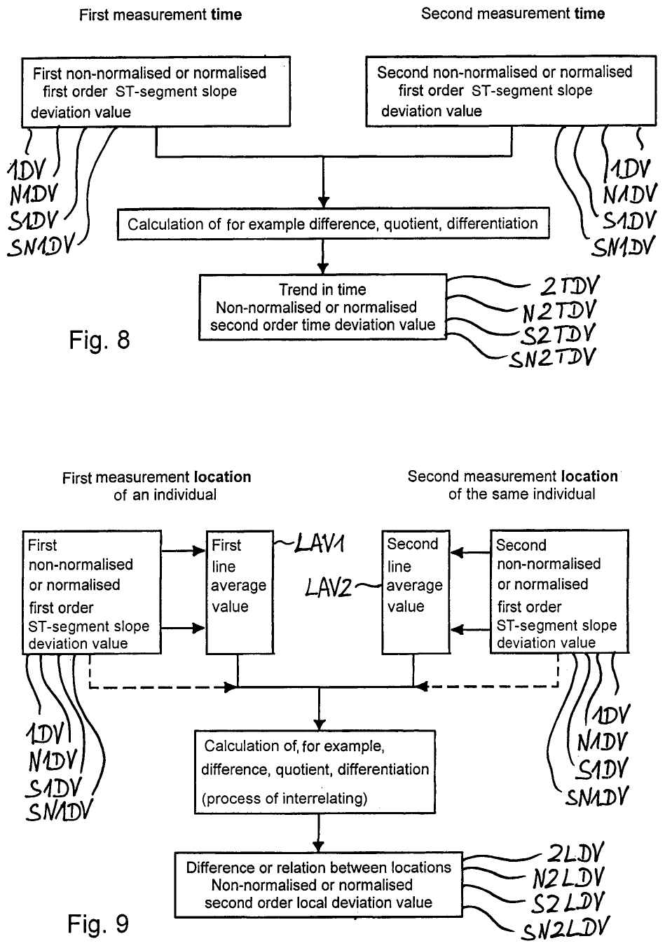

In order to calculate normalized deviation values N1DV from the ST-segment slope deviation values 1DV, the ST-segment slope deviation values 1DV is related to a ST-segment slope characteristic value CV which is derived from ST-segment slope values VX. This is noted as a process of relating in FIG. 7 Preferably, a division is carried out with the first order ST-segment slope value 1DV as the dividend and the ST segment slope characteristic value CV as the divisor. The result of the relating process, i.e. the quotient in the case shown in FIG. 7 is a normalized ST-segment slope deviation values N1DV. It is also possible to carry out this relating processes with second order ST-segment slope values which are shown in FIGS. 8 and 9. Preferably, the ST-segment slope characteristic value CV is a mean value, an average value, or an expected value of ST-segment slope values VX. Optionally, also the ST-segment slope values V1 and V2 of which the first order deviation values 1DV have been derived can be basis of the ST-segment slope characteristic value CV. The normalized ST-segment slope deviation values N1DV can be used as assessment data. It is also possible to renounce of the normalization. Then, the T-wave deviation values 1DV can be used as assessment data. The latter is for example possible, if data are used from one single individual only. For example, a change during measurement time of a ST-segment slope deviation value measured from one source of a human does not necessarily need normalization in order to assess a risk of sudden cardiac death, for example. However, it is preferred to have a universal arrhythmia assessment system with normalization which can used on every individual of a species. Then, fixed numbers can be used for assessment or diagnosis of a condition independently for every patient.

As shown in FIG. 7, also a statistical enhancement of the quality of the ST-segment slope deviation value 1DV or the normalized ST-segment slope deviation N1DV value can be carried out. More than one of these values, respectively, can be processed statistically to obtain a an average value, a mean value, an expected value or a median, in general, a value that represents relevant information of more than one of these values regarding a general magnitude. Using the statistical values S1DV, SN1DV instead of non-statistical values can enhance assessment and/or diagnosis quality. It is also possible to carry out the normalization before the relating process is carried out which is not shown in FIG. 7. Then, the statistical ST-segment slope deviation value S1DV is related to, particularly divided by, the characteristic value CV to obtain the normalized statistical ST-segment slope deviation value SN1DV.

FIG. 8 schematically shows an information flow from non-normalized or normalized, statistical or non-statistical ST-segment slope deviation values 1DV, N1DV, S1DV, SN to a corresponding second order time deviation value 2TDV, N2TDV, S2TDV, SN2TDV. In order to find a trend of a type of first order ST-segment slope deviation value 1DV, N1DV, S1DV, SN1DV, two values of a type of ST-segment slope deviation values 1DV, N1DV, S1DV, SN which have been taken at different times can be compared to each other, especially subtracted from each other or divided one by the other, to derive a second order time deviation value 2TDV, N2TDV, S2TDV, SN2TDV. The second order time deviation value 2TDV, N2TDV is a single value comprising information of how much the type of T-wave deviation value 1DV, N1DV, S1DV, SN changes in time. For example, an event time period and/or an event point of time and/or based on this, a risk of a sudden cardiac death or an arrhythmogenic right ventricular cardiomyopathy can be assessed or diagnosed.

FIG. 9 schematically shows an information flow from non-normalized or normalized, statistical or non-statistical ST-segment slope deviation values 1DV, N1DV, S1DV, SN to a second order local ST-segment slope deviation value 2LDV, N2LDV, S2LDV, SN2DLV. In order to find a local deviation of a type of a first order ST-segment slope deviation value 1DV, N1DV, S1DV, SN1DV, ST-segment slope deviation values 1DV, N1DV, S1DV, SN1DV of the same type which have been measured at different locations and which are thus influenced by different parts of the myocardium can be interrelated. Particularly, a first and a second of such values are subtracted from each other or divided one by the other, to derive a second order local deviation value 2LDV, N2DLV S2LDV, SN2DLV, respectively. This value comprises information of how much the first order T-wave deviation value 1DV, N1DV, S1DV, SN1DV changes in regard of the location of measurement. In the local deviation, a special type of information is present, as it correlates, in dependency of its magnitude, with the conditions J) to K) in FIG. 6.

It is possible to derive a second order local ST-segment slope deviation value 2LDV, N2DLV S2LDV, SN2DLV from a single or at least two first order local ST-segment slope deviation values 1DV, N1DV, S1DV, SN1DV. The latter case is represented in FIG. 9 by two arrows per first order value pointing at a first and a second line average value LAV1, LAV2, respectively. By a line average value LAV1, LAV2, one location can be represented by more than one first order ST-segment slope deviation value 1DV, N1DV, S1DV, SN1DV. These first order ST-segment slope deviation values 1DV, N1DV, S1DV, SN1DV can be combined or merged, for example statistically, to obtain one single line average value LAV1, LAV2 representing a location. Preferably, values that originate from at least two different lines which are neighbors to each other are merged to a line average value. It is also possible that the whole heart is considered a location and thus can be compared to another location which is part of this heart. For example, the whole heart can be represented by a line average value LAV1, LAV2 that is calculated from all or a representative choice of the lines.

The interrelation can be carried out with single first order statistical or non-statistical ST-segment slope deviation values 1DV, N1DV, S1DV, SN which is shown by the dotted line arrows in FIG. 9. Alternatively or additionally, the interrelation can be carried out with two different line average values LAV. It is also possible to interrelate a line average values LAV and a single first order statistical or non-statistical ST-segment slope deviation value 1DV, N1DV, S1DV, SN1DV of different locations.

In a preferred embodiment, an information flow that is shown in FIGS. 6 to 9 is realised.

1. Data processing apparatus (12) for processing ST-segment slope information (ST1, ST2, STX) from an ECG signal from a myocardium of a heart of a human (1) or animal to generate data for one of
an assessment of a risk, a probability or a presence and
a diagnosis of a risk, a probability or a presence
of a type of arrhythmia of the heart or a part of it,
characterized in that the data processing apparatus (12) is configured
to calculate at least two ST-segment slope values (STS1, STS2, STSX) wherein each of the at least two ST-segment slope values (STS1, STS2, STSX) belongs to ST-segments (ST) from different heart beats (HB1, HB2, HBX) of the same heart, and
to derive a ST-segment slope deviation value (1DV, N1DV, S1DV, SN1DV) from at least two ST-segment slope values (STS1, STS2, STSX).

2. Data processing apparatus (12) according to claim 1, characterized in that the ST-segment slope information (ST1, ST2, STX) for obtaining the ST-segment slope value (STS1, STS2, STSX) has a resolution of less than 5 ms, preferably less than 285 μs, preferably less than 100 μs and most preferably less than 50 μs in time and/or of less than 50 mV preferably less than 16 μV in amplitude, preferably less than 1 μV and most preferable less than 1 nV.

3. Data processing apparatus (12) according to claim 1 or 2, characterized in that the ST-segment slope value (STS1, STS2, STSX) from an ECG signal (ECG) is
A) an angle (α) between
the ST-segment (ST) or a line that is at least approximately parallel to ST-segment (ST) or a characteristic part of one of them, particularly and
a line or a value in an ECG diagram (ECG) with an angle, which is at least approximately constant during time, particularly a zeroline (Z), and/or
B) an angle (α) between
the ST-segment (ST) or a line that is at least approximately parallel to ST-segment (ST) or a characteristic part of one of them, particularly and
another part of PQRST-complex which, preferably, repeats in heart beats with at least approximately the same angle in respect to time axis, particularly TP-segment,
C) a ratio of an amplitude change to a time change between two points in a ST-segment (ST) or of a line that is at lest approximately parallel to ST-segment (ST), preferably distinct points, and/or
D) a derivative in respect of time of the ST-segment (ST) or a line that is at least approximately parallel to ST-segment (ST) or of a part of one of them, and/or
E) a median value of at least two ST-segment slope values STS1, STS2, STSX) taken from parts of one single ST-segment (ST) or a line that is at lest approximately parallel to it,
F) a difference between two points of ST-segment (ST) or a line that is at least approximately parallel to it, preferably in a known time distance (at) to each other or at characteristic points in the ST segment (ST),
G) a difference between or a ratio of two function values of injective or bijective functions of which the the argument is a value of ST-segment (ST) or a line that is at least approximately parallel to it, respectively, preferably in a known time distance (Δt) to each other or at characteristic points in the ST segment (ST), wherein the function preferably is a power and most preferably a square function,
H) a value obtained from an approximation or matching of the slope of ST-segment (ST) or of a line that is at lest approximately parallel to it or a part of one of them, in which the ST-segment slope information (ST1, ST2, STX) is approximated or described with a mathematical function or approximation or description data and calculated thereof as a ST-segment slope value (STS1, STS2, STSX) according to one of the items A) to G),
wherein, preferably, the ST-segment slope value (STS1, STS2, STSX) is provided for assessment or diagnosis of a type of arrhythmia of a heart or a part of it.

4. Data processing apparatus (12) according to one of the preceding claims, characterised in that two ST-segment slope values (STS1, STS2, STSX) are compared to each other to derive a ST-segment slope deviation value (1DV, N1DV, S1DV, SN1DV), which preferably is a mathematical difference between the two ST-segment slope values (STS1, STS2, STSX) or a quotient of the two ST-segment slope values (STS1, STS2, STSX).

5. Data processing apparatus (12) according to one of the preceding claims, characterized in that the data processing apparatus (12) is configured to calculate a normalized ST-segment slope deviation value (N1DV, SN1DV) by relating
the ST-segment slope deviation value (1DV, S1DV) to
a characteristic ST-segment slope value (CV) calculated from ST-segment slope values (STS1, STS2, STSX) from the same heart from which the ST-segment slope deviation value has been derived,
wherein, particularly, ST-segment slopes (ST) for calculation of the characteristic value (CV) are determined from same measurement location at the same heart as the ST-segment slope deviation value (1DV, S1DV), and more particularly from the same lead (3) as the ST-segment slope deviation value (1DV, S1DV).

6. Data processing apparatus (12) according to one of the preceding claims, characterized in that the data processing apparatus (12) is configured to carry out a statistical method on a plurality of ST-segment slope values (STS1, STS2, STSX) or non-normalized or normalized ST-segment slope deviation values (1DV, N1DV) to obtain a non-normalized or normalized statistical ST-segment slope deviation value (S1DV, SN1DV) that represents the variability or dispersion of the ST-segment slope angle (α) of different heartbeats (HB1, HB2, HBX),
wherein, preferably, a statistical ST-segment slope deviation value (S1DV, SN1DV) is calculated by statistics of the ST-segment slope deviation values (1DV, N1DV) over time, wherein the ST-segment slope deviation values (1DV, N1DV) preferably originate from the the same source,
wherein the variability or dispersion is preferably calculated as a standard deviation, a variance or a mean value of deviations between ST-segment slope values, especially deviations of ST-segment slope values (1DV, N1DV) that are neighbors in time.

7. Data processing apparatus (12) according to one of the preceding claims, characterized in that the data processing apparatus (12) is configured
to interrelate a first
ST-segment slope deviation value (1DV) or
normalized ST-segment slope deviation value (N1DV) or
statistical ST-segment slope deviation values (S1DV) or
statistical normalized ST-segment slope deviation value (SN1DV),
non-normalized line average value calculated from one or more ST-segment slope deviation values (1DV) and/or one or more statistical ST-segment slope deviation values (S1DV), preferably calculated of the same value type, or
normalized line average value calculated from one or more normalized ST-segment slope deviation values (N1DV) and/or one or more normalized statistical ST-segment slope deviation values (SN1DV), preferably calculated of the same value type,
of at least one first source representing a first location at the heart of a human (1) or animal, wherein an averaged value represents two or more first sources at the first location,
to a second
ST-segment slope deviation value (1DV) or
normalized ST-segment slope deviation value (N1DV) or
statistical ST-segment slope deviation value (S1DV) or
statistical normalized ST-segment slope deviation value (SN1DV)
non-normalized line average value calculated from one or more ST-segment slope deviation values (1DV) and/or one or more statistical ST-segment slope deviation values (S1DV), preferably calculated of the same value type, or
normalized line average value calculated from one or more normalized ST-segment slope deviation values (N1DV) and/or one or more normalized statistical ST-segment slope deviation values (SN1DV), preferably calculated of the same value type,
of at least one second source representing a second location at the heart of a human (1) or animal, wherein an averaged value represents two or more second sources at the second location,
wherein the second location represents
a great part of the heart or the whole heart, or
a part of the heart that is different from the first location, or
the whole heart except for the first location,
and to generate a second-order local deviation value (2LDV, S2LDV) or a normalized second-order local deviation value (N2DV, SN2DV),
wherein, preferably, the first and the second value is of the same type and/or the process of interrelating is a subtraction, a division or a mathematical derivation or a statistical process.

8. Data processing apparatus (12) according to one of the preceding claims, characterized in that the data processing apparatus (12) is configured to
process, from the same heart, a number of at least 2 ST-segment slopes, preferably at least 60 ST-segment slopes and most preferably about 200 ST-segment slopes and/or process a number of ST-segment slopes of a PQRST-complex during at least 1 second, preferably at least 2, 3 or 4 seconds, more preferably at least 30 seconds and most preferably about 200 seconds to obtain ST-segment slope deviation values or normalized ST-segment slope deviation values.

9. Data processing apparatus (12) to one of the preceding claims, characterized in that the data processing apparatus (12) is configured
to compare a first ST-segment slope deviation value (1DV) or a first normalized ST-segment slope deviation value (N1DV) or a first statistical ST-segment slope deviation value (S1DV) or a first statistical normalized ST-segment slope deviation value (SN1DV) measured at a first time
to a second ST-segment slope deviation value (1DV) or a second normalized ST-segment slope deviation value (N1DV) or a second statistical ST-segment slope deviation value (S1DV) or a second statistical normalized ST-segment slope deviation value (SN1DV), preferably of the same type of value as first value, measured at a second time that is different from the first time and
to generate a second order time deviation value (2TDV) or normalized second order time deviation value (N2TDV) or a statistical second order time deviation value (S2TDV) or statistical normalized second order time deviation value (SN2TDV) which is based on the comparison.

10. Data processing apparatus (12) according to one of the preceding claims, characterized in that the data processing apparatus (12) comprises a measurement device (11) for generating ST-segment slope information (ST1, ST2, STX) from an individual human (1) or animal by measurement,
wherein the measurement device (11) preferably has a resolution of less than 5 ms, preferably less than 285 μs, preferably less than 100 μs and most preferably less than 50 μs in time and/or of less than 50 mV preferably less than 16 μV in amplitude, preferably less than 1 μV and most preferable less than 1 nV.

11. Data processing apparatus (12) according to one of the preceding claims, characterized in that the data processing apparatus (12) comprises a value signalling device which is configured to signal and/or display a ST-segment slope deviation value (1DV) and/or normalized ST-segment slope deviation value (N1DV) and/or a statistical ST-segment slope deviation value (S1DV) and/or a statistical normalized ST-segment slope deviation value (SN1DV) and/or a second order ST-segment slope deviation value (2TDV, 2LDV) and/or a second order normalized ST-segment slope deviation value (N2TDV, N2LDV) and/or a statistical second order ST-segment slope deviation value (S2TDV, S2LDV) and/or a statistical second order normalized ST-segment slope deviation value (SN2TDV, SN2LDV) to a person or to transfer corresponding data to another unit as the data processing apparatus (12).

12. Assessment apparatus (14) for an assessment of risk and/or presence and/or extent of arrhythmia of a heart or a part of it of an individual human (1) or animal, characterized in that the assessment apparatus (14) is configured to base an assessment or a diagnosis on at least one a ST-segment slope deviation value (1DV) and/or normalized ST-segment slope deviation value (N1DV) and/or statistical ST-segment slope deviation value (S1DV) and/or statistical normalized ST-segment slope deviation value (SN1DV) and/or second order ST-segment slope deviation value (2TDV, 2LDV) and/or second order normalized ST-segment slope deviation value (N2TDV, N2LDV) and/or statistical second order ST-segment slope deviation value (S2TDV, S2LDV) and/or statistical second order normalized ST-segment slope deviation value (SN2TDV, SN2LDV) provided by a data processing apparatus (12) according to one of claims 1 to 11 to assess or diagnose a condition of at least a part of a myocardium and preferably of a heart.

13. Assessment apparatus (14) according to claim 12, characterized in that the assessment apparatus (14) is configured to assess at least one of the following types of arrhythmia
A) risk and/or presence and/or extent of sick sinus syndrome and/or sinoatrial node dysfunction and/or atrioventricular heart block, and/or
B) risk and/or presence and/or extent of sinus extrasystole and/or atrial extrasystole, and/or atrioventricular extrasystole, and/or
C) risk and/or presence and/or extent of supraventricular tachycardia, and/or
D) risk and/or presence and/or extent of atrial flutter, and/or
E) risk and/or presence and/or extent of paroxysmal atrial fibrillation, and/or
F) risk and/or presence and/or extent of atrial fibrillation, and/or
G) risk and/or presence and/or extent of ventricular tachycardia, and/or
H) risk of ventricular fibrillation, and/or
I) risk of sudden cardiac death
by an assessment value that is
a ST-segment slope deviation value (1DV) and/or
a normalized ST-segment slope deviation value (N1DV) and/or
a statistical ST-segment slope deviation value (S1DV) and/or
a statistical normalized ST-segment slope deviation value (SN1DV) and/or
an average value of ST-segment slope deviation values (1DV) and/or
an average value of normalized ST-segment slope deviation values (N1DV) and/or
an average value of statistical ST-segment slope deviation values (S1DV) and/or
an average value of statistical normalized ST-segment slope deviation values (SN1DV),
wherein an average value represents at least one location at the heart, and/or
J) risk and/or presence and/or extent of bundle branch block, and/or
K) risk and/or presence and/or extent of ventricular premature beats and/or polymorphic ventricular premature beats, and/or
L) risk and/or presence and/or extent of arrhythmogenic right ventricular cardiomyopathy and/or dysplasia and/or its complications
by an assessment value that is a result of a n interrelation of a first
ST-segment slope deviation value (1DV) or
normalized ST-segment slope deviation value (N1DV) or
statistical ST-segment slope deviation values (S1DV) or
statistical normalized ST-segment slope deviation value (SN1DV),
non-normalized line average value calculated from one or more ST-segment slope deviation values (1DV) and/or one or more statistical ST-segment slope deviation values (S1DV), preferably calculated of the same value type, or
normalized line average value calculated from one or more normalized ST-segment slope deviation values (N1DV) and/or one or more normalized statistical ST-segment slope deviation values (SN1DV), preferably calculated of the same value type,
of at least one first source representing a first location at the heart of a human (1) or animal, wherein an averaged value represents two or more first sources at the first location,
to a second
ST-segment slope deviation value (1DV) or
normalized ST-segment slope deviation value (N1DV) or
statistical ST-segment slope deviation value (S1DV) or
statistical normalized ST-segment slope deviation value (SN1DV)
non-normalized line average value calculated from one or more ST-segment slope deviation values (1DV) and/or one or more statistical ST-segment slope deviation values (S1DV), preferably calculated of the same value type, or
normalized line average value calculated from one or more normalized ST-segment slope deviation values (N1DV) and/or one or more normalized statistical ST-segment slope deviation values (SN1DV), preferably calculated of the same value type,
of at least one second source representing a second location at the heart of a human (1) or animal, wherein an averaged value represents two or more second sources at the second location,
wherein the second location represents
a great part of the heart or the whole heart, or
a part of the heart that is different from the first location, or
the whole heart except for the first location,
and to generate a second-order local deviation value (2LDV, S2LDV) or a normalized second-order local deviation value (N2DV, SN2DV),
wherein, preferably, the first and the second value is of the same type and/or the process of interrelating is a subtraction, a division or a mathematical derivation or a statistical process,
wherein, preferably, by the magnitude of one of the assessment values, at least one of the risk and/or presences and/or extents of one of the diseases A) to I) is assessed and/or
M) an event time period or an event point in time until a beginning of an arrhythmia of a heart or a part of it as mentioned in item A) to L)
by extrapolation in time using a second order ST-segment slope time deviation value (2TDV, S2TDV) or normalized second order ST-segment slope time deviation value (N2TDV, SN2TDV), and/or
by a speed of decreasing or increasing in time of a non-statistical or statistical second order local ST-segment slope deviation value (2LDV, S2LDV) or a non-statistical or statistical normalized second order local ST-segment slope deviation value (N2LDV, SN2LDV) and/or a line average value of non-statistical or statistical ST-segment slope deviation value (1DV, S1DV) or a line average value of non-statistical or statistical normalized ST-segment slope deviation values (N1DV, SN1DV),
wherein, preferably, the speed of decreasing or increasing is calculated from two or more
non-statistical or statistical ST-segment slope deviation values (1DV, S1DV) or
non-statistical or statistical normalized ST-segment slope deviation values (N1DV, SN1DV) or
line average values of non-statistical or statistical ST-segment slope deviation values (1DV, S1DV) or
line average values of non-statistical or statistical normalized ST-segment slope deviation values (N1DV, SN1DV) or
non-statistical or statistical second order local deviation values (2LDV, S2LDV) or
non-statistical or statistical normalized second order local deviation values (N2LDV, SN2LDV)
and a time period between the points in time at which the respective deviation values were detected.

14. Assessment apparatus (14) according to claim 12, characterized in that the assessment apparatus (14) is configured to carry out a comparison of assessment information to a threshold value in order to
make a diagnosis proposal and/or
make an automatic diagnosis and/or
automatically notify a medical service
when a decision about a predefined risk, presence or extent of the arrhythmias A) to L) or an event time period or an event point in time M) has been made,
wherein preferably the assessment apparatus (14) comprises a diagnosis signaling device for signaling a diagnosis proposal or an automatic diagnosis of an arrhythmia or disease A) to L) or an event time period or an event point in time M) to a person or to transfer corresponding data to another unit as the assessment apparatus (14).

15. Method for providing data for an assessment and/or a diagnosis of a condition or a disease of at least a part of the myocardium or the whole myocardium of a heart of a human (1) or an animal, wherein the method includes processing ST-segment slope information (ST1, ST2, STX) from a signal from a myocardium of a heart of a human (1) or animal, characterised in that the method includes deriving a ST-segment slope deviation value (1DV) from at least one deviation between at least two ST-segment slope values (STS1, STS2, STSX) of the same type which originate from different heartbeats of the heart in order to generate assessment data (22).

16. Method for assessing a condition or a disease of the heart of a human (1) or animal, characterised in that least one of the following assessments is carried out with non-statistical or statistical first and/or second order deviation values (1DV, S1DV, 2LDV, S2LDV) and/or first and/or second order normalized deviation values (N1DV, NS1DV, N2LDV, SN2LDV) and/or averaged first and/or second order deviation values and/or averaged first and/or second order normalized deviation values which are provided by a data processing apparatus according to claims 1 to 10 and/or by a method for providing data according to claim 14:
A) risk and/or presence and/or extent of sick sinus syndrome and/or sinoatrial node dysfunction and/or atrioventricular heart block, and/or
B) risk and/or presence and/or extent of sinus extrasystole and/or atrial extrasystole, and/or atrioventricular extrasystole, and/or
C) risk and/or presence and/or extent of supraventricular tachycardia, and/or
D) risk and/or presence and/or extent of atrial flutter, and/or
E) risk and/or presence and/or extent of paroxysmal atrial fibrillation, and/or
F) risk and/or presence and/or extent of atrial fibrillation, and/or
G) risk and/or presence and/or extent of ventricular tachycardia, and/or
H risk of ventricular fibrillation, and/or
I) risk of sudden cardiac death
by an assessment value that is
a ST-segment slope deviation value (1DV) and/or
a normalized ST-segment slope deviation value (N1DV) and/or
a statistical ST-segment slope deviation value (S1DV) and/or
a statistical normalized ST-segment slope deviation value (SN1DV) and/or
an average value of ST-segment slope deviation values (1DV) and/or
an average value of normalized ST-segment slope deviation values (N1DV) and/or
an average value of statistical ST-segment slope deviation values (S1DV) and/or
an average value of statistical normalized ST-segment slope deviation values (SN1DV),
wherein an average value represents at least one location at the heart, and/or
J) risk and/or presence and/or extent of bundle branch block, and/or
K) risk and/or presence and/or extent of ventricular premature beats and/or polymorphic ventricular premature beats, and/or
L) risk and/or presence and/or extent of arrhythmogenic right ventricular cardiomyopathy and/or dysplasia and/or its complications by an assessment value that is a result of a n interrelation of a first
ST-segment slope deviation value (1DV) or
normalized ST-segment slope deviation value (N1DV) or
statistical ST-segment slope deviation values (S1DV) or
statistical normalized ST-segment slope deviation value (SN1DV),
non-normalized line average value calculated from one or more ST-segment slope deviation values (1DV) and/or one or more statistical ST-segment slope deviation values (S1DV), preferably calculated of the same value type, or
normalized line average value calculated from one or more normalized ST-segment slope deviation values (N1DV) and/or one or more normalized statistical ST-segment slope deviation values (SN1DV), preferably calculated of the same value type,
of at least one first source representing a first location at the heart of a human (1) or animal, wherein an averaged value represents two or more first sources at the first location,
to a second
ST-segment slope deviation value (1DV) or
normalized ST-segment slope deviation value (N1DV) or
statistical ST-segment slope deviation value (S1DV) or
statistical normalized ST-segment slope deviation value (SN1DV)
non-normalized line average value calculated from one or more ST-segment slope deviation values (1DV) and/or one or more statistical ST-segment slope deviation values (S1DV), preferably calculated of the same value type, or
normalized line average value calculated from one or more normalized ST-segment slope deviation values (N1DV) and/or one or more normalized statistical ST-segment slope deviation values (SN1DV), preferably calculated of the same value type,
of at least one second source representing a second location at the heart of a human (1) or animal, wherein an averaged value represents two or more second sources at the second location,
wherein the second location represents
a great part of the heart or the whole heart, or
a part of the heart that is different from the first location, or
the whole heart except for the first location,
and to generate a second-order local deviation value (2LDV, S2LDV) or a normalized second-order local deviation value (N2DV, SN2DV),
wherein, preferably, the first and the second value is of the same type and/or the process of interrelating is a subtraction, a division or a mathematical derivation or a statistical process,
wherein, preferably, by the magnitude of one of the assessment values, at least one of the risk and/or presences and/or extents of one of the diseases A) to I) is assessed and/or
N) an event time period or an event point in time until a beginning of an arrhythmia of a heart or a part of it as mentioned in item A) to L)
by extrapolation in time using a second order ST-segment slope time deviation value (2TDV, S2TDV) or normalized second order ST-segment slope time deviation value (N2TDV, SN2TDV), and/or
by a speed of decreasing or increasing in time of a non-statistical or statistical second order local ST-segment slope deviation value (2LDV, S2LDV) or a non-statistical or statistical normalized second order local ST-segment slope deviation value (N2LDV, SN2LDV) and/or a line average value of non-statistical or statistical ST-segment slope deviation value (1DV, S1DV) or a line average value of non-statistical or statistical normalized ST-segment slope deviation values (N1DV, SN1DV),
wherein, preferably, the speed of decreasing or increasing is calculated from two or more
non-statistical or statistical ST-segment slope deviation values (1DV, S1DV) or
non-statistical or statistical normalized ST-segment slope deviation values (N1DV, SN1DV) or
line average values of non-statistical or statistical ST-segment slope deviation values (1DV, S1DV) or
line average values of non-statistical or statistical normalized ST-segment slope deviation values (N1DV, SN1DV) or
non-statistical or statistical second order local deviation values (2LDV, S2LDV) or
non-statistical or statistical normalized second order local deviation values (N2LDV, SN2LDV)
and a time period between the points in time at which the respective deviation values were detected.
